(19) United States
(12) Patent Application Publication
(10) Pub. No.: US 2008/0003667 A1
(43) Pub. Date: Jan. 3, 2008

(54) CONSUMABLE ELEMENTS FOR USE WITH FLUID PROCESSING AND DETECTION SYSTEMS

(75) Inventors: Mark Jones, Reading, MA (US); Christopher Petroff, Groton, MA (US); Dan Liu, Sunnyvale, CA (US)

Correspondence Address: AFFYMETRIX, INC;ATTN: CHIEF IP COUNSEL, LEGAL DEPT., 3420 CENTRAL EXPRESSWAY, SANTA CLARA, CA, 95051, US

(73) Assignee: Affymetrix, Inc., Santa Clara, CA (US)

(21) Appl. No.: 11/733,847

(22) Filed: Apr. 11, 2007

Related U.S. Application Data
(60) Provisional application No. 60/747,690, filed on May 19, 2006.
(60) Provisional application No. 60/823,702, filed on Aug. 28, 2006.

(51) Int. Cl. C12M 1/34

(52) U.S. Cl. 435/287.2

(57) ABSTRACT

One embodiment describes an automated and flexible system to analyze probe arrays. It comprises a plurality of arrays mounted on pegs that are moved by an instrument handling robot to liquid reaction stations.

### PRIORITY CLAIM

The present application claims priority to U.S. Provisional Patent Application No. 60/747,690 filed on May 19, 2006; and 60/823,702 filed on Aug. 28, 2006, the entire disclosure of which are incorporated herein by reference in their entireties for all purposes.

### BACKGROUND

1. Field of the Invention

The present invention relates to systems and methods for examining biological material. In particular, the invention relates to consumable elements for use with a system for fluid processing and image acquisition instruments, where the fluid processing instruments performs the steps of hybridization, washing, and staining biological probe arrays in preparation for scanning.

2. Related Art

Synthesized nucleic acid probe arrays, such as Affymetrix GeneChip® probe arrays, and spotted probe arrays, have been used to generate unprecedented amounts of information about biological systems. For example, the GeneChip® Human Genome U133 Plus 2.0 Array available from Affymetrix, Inc. of Santa Clara, Calif., is comprised of one microarray containing 1,300,000 oligonucleotide features covering more than 47,000 transcripts and variants that include 38,500 well characterized human genes. Further, the GeneChip® Mapping 500K Array Set available from Affymetrix, Inc. of Santa Clara, Calif., is comprised of two microarrays capable of genotyping on average 250,000 SNPs per array. Analysis of expression and genotype data from such microarrays may lead to the development of new drugs and new diagnostic tools.

### SUMMARY OF THE INVENTION

Systems, methods, and products to address these and other needs are described herein with respect to illustrative, non-limiting, implementations. Various alternatives, modifications and equivalents are possible. For example, certain systems, methods, and computer software products are described herein using exemplary implementations for analyzing data from arrays of biological materials such as, for instance, Affymetrix® GeneChip® probe arrays. However, these systems, methods, and products may be applied with respect to many other types of probe arrays and, more generally, with respect to numerous parallel biological assays produced in accordance with other conventional technologies and/or produced in accordance with techniques that may be developed in the future. For example, the systems, methods, and products described herein may be applied to parallel assays of nucleic acids, PCR products generated from cDNA clones, proteins, antibodies, or many other biological materials. These materials may be disposed on slides (as typically used for spotted arrays), on substrates employed for GeneChip® arrays, or on beads, optical fibers, or other substrates or media, which may include polymeric coatings or other layers on top of slides or other substrates. Moreover, the probes need not be immobilized in or on a substrate, and, if immobilized, need not be disposed in regular patterns or arrays. For convenience, the term “probe array” will generally be used broadly hereafter to refer to all of these types of arrays and parallel biological assays.

One embodiment of the present invention is an automated and flexible system for conducting hybridization experiments comprising an automated robot for moving a strip to selected location; a plurality of pegs holding nucleic acid arrays removably mounted on a strip; at least one tray containing wells in rows, the wells having reagents to interact with an array on the peg, each row being associated with a different process step; and a system to align the peg with the wells on the tray. Other preferred embodiments comprise a system where the strip has four pegs, it is removably mounted to a plate, encased, it contains a bar code reader to identify strips or trays, and it may be operatively connected to a hybridization station and scanning station.

In another embodiment, there is a system to align the pegs with the wells on the tray, comprising; a set of fiducial features on a operatively connected to the tray; and a set of sensors operatively connected to the automated robot to determine a position for the tray relative to the robot.

Another embodiment of the invention discloses a method for controlling the temperature of a wash solution comprising: a heater block whose shape is tailored to give the same temperature in all the wells; a temperature probe to measure the heater block temperature; a temperature probe to measure the room temperature; and a calibrated look up table to adjust for differences between the heater block temperature and the temperature of liquid in the wells based on room temperature.

The above embodiments and implementations are not necessarily inclusive or exclusive of each other and may be combined in any manner that is non-conflicting and otherwise possible, whether they be presented in association with a same, or a different, embodiment or implementation. The description of one embodiment or implementation is not intended to be limiting with respect to other embodiments and/or implementations. Also, any one or more function, step, operation, or technique described elsewhere in this specification may, in alternative implementations, be combined with any one or more function, step, operation, or technique described in the summary. Thus, the above embodiment and implementations are illustrative rather than limiting.

### BRIEF DESCRIPTION OF THE DRAWINGS

The above and further features will be more clearly appreciated from the following detailed description when taken in conjunction with the accompanying drawings. In the drawings, like reference numerals indicate like structures or method steps and the leftmost digit of a reference numeral indicates the number of the figure in which the referenced element first appears (for example, the element 100 appears first in FIG. 1). In functional block diagrams, rectangles generally indicate functional elements and parallelograms generally indicate data. In method flow charts, rectangles generally indicate method steps and diamond shapes generally indicate decision elements. All of these conventions, however, are intended to be typical or illustrative, rather than limiting.

FIG. 1 is a functional block diagram of one embodiment of a fluid processing instrument and scanner system in communication with a computer system for processing and acquiring data from one or more probe arrays;

FIG. 2 is a functional block diagram of one embodiment of the fluid processing instrument and computer of FIG. 1, including fluid processing system components that comprise compartments and internal robotic manipulator;

FIG. 3 is a functional block diagram of one embodiment of the fluid processing instrument of FIG. 2 comprising a wash station, a hyb station, and a scan prep station;

FIG. 4 is a simplified graphical example of one embodiment of the fluid processing instrument of FIGS. 2 and 3 comprising a strip with multiple probe arrays and a drawer comprising the stations of FIG. 3;

FIG. 5A is a simplified graphical example of one embodiment of the strip with multiple probe arrays of FIG. 4;

FIG. 5B is a simplified graphical example of one embodiment of the drawer comprising the stations of FIG. 4;

FIG. 6A is a simplified graphical example of one embodiment of the wash station of FIG. 3 comprising a wash tray; and

FIG. 6B is a simplified graphical representation of one embodiment of a multiple use tray.

FIG. 7 is an example of a scan tray to hold peg strips while scanning.

FIG. 8 is an example of a plate adapter to hold individual pegs.

FIGS. 9A, 9B, 10A, 10B, and 10C show different views of a device to hold peg strips.

FIG. 11 is an example of two pair of discrete optoelectronic sensors positioned around a fiducial pin.

FIG. 12 is an example of a gripper element as it approaches the fiducial pins on a support or drawer for holding well containers.

### DETAILED DESCRIPTION

The description below is designed to present specific embodiments and not to be construed as limiting in any way. Also, reference will be made to articles and patents to show general features that are incorporated into the present disclosure, but the invention is not limited by these descriptions.

### a) General

The present invention has many preferred embodiments and relies on many patents, applications and other references for details known to those of the art. Therefore, when a patent, application, or other reference is cited or repeated below, it should be understood that it is incorporated by reference in its entirety for all purposes as well as for the proposition that is recited.

As used in this application, the singular form “a,” “an,” and “the” include plural references unless the context clearly dictates otherwise. For example, the term “an agent” includes a plurality of agents, including mixtures thereof.

An individual is not limited to a human being but may also be other organisms including but not limited to mammals, plants, bacteria, or cells derived from any of the above.

Throughout this disclosure, various aspects of this invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

The practice of the present invention may employ, unless otherwise indicated, conventional techniques and descriptions of organic chemistry, polymer technology, molecular biology (including recombinant techniques), cell biology, biochemistry, and immunology, which are within the skill of the art. Such conventional techniques include polymer array synthesis, hybridization, ligation, and detection of hybridization using a label. Specific illustrations of suitable techniques can be had by reference to the example herein below. However, other equivalent conventional procedures can, of course, also be used. Such conventional techniques and descriptions can be found in standard laboratory manuals such as Genome Analysis: A Laboratory Manual Series (Vols. I-IV), Using Antibodies: A Laboratory Manual, Cells: A Laboratory Manual, PCR Primer: A Laboratory Manual, and Molecular Cloning: A Laboratory Manual (all from Cold Spring Harbor Laboratory Press), Stryer, L. (1995) Biochemistry (4th Ed.) Freeman, N.Y., Gait, “Oligonucleotide Synthesis: A Practical Approach” 1984, IRL Press, London, Nelson and Cox (2000), Lehninger, Principles of Biochemistry 3rd Ed., W.H. Freeman Pub., New York, N.Y. and Berg et al. (2002) Biochemistry, 5^{th} Ed., W.H. Freeman Pub., New York, N.Y., all of which are herein incorporated in their entirety by reference for all purposes.

The present invention can employ solid substrates, including arrays in some preferred embodiments. Methods and techniques applicable to polymer (including protein) array synthesis have been described in U.S. Ser. No. 09/536,841, WO 00/58516, U.S. Pat. Nos. 5,143,854, 5,242,974, 5,252,743, 5,324,633, 5,384,261, 5,405,783, 5,424,186, 5,451,683, 5,482,867, 5,491,074, 5,527,681, 5,550,215, 5,571,639, 5,578,832, 5,593,839, 5,599,695, 5,624,711, 5,631,734, 5,795,716, 5,831,070, 5,837,832, 5,856,101, 5,858,659, 5,936,324, 5,945,334, 5,968,740, 5,974,164, 5,981,185, 5,981,956, 6,025,601, 6,033,860, 6,040,193, 6,090,555, 6,136,269, 6,269,846 and 6,428,752, in PCT Applications Nos. PCT/US99/00730 (International Publication Number WO 99/36760) and PCT/US01/04285 (International Publication Number WO 01/58593), which are all incorporated herein by reference in their entirety for all purposes.

Patents that describe synthesis techniques in specific embodiments include U.S. Pat. Nos. 5,412,087, 6,147,205, 6,262,216, 6,310,189, 5,889,165, and 5,959,098. Nucleic acid arrays are described in many of the above patents, but the same techniques are applied to polypeptide arrays.

Nucleic acid arrays that are useful in the present invention include those that are commercially available from Affymetrix (Santa Clara, Calif.) under the brand name GeneChip®. Example arrays are shown on the website at affymetrix.com.

The present invention also contemplates many uses for polymers attached to solid substrates. These uses include gene expression monitoring, profiling, library screening, genotyping and diagnostics. Gene expression monitoring and profiling methods can be shown in U.S. Pat. Nos. 5,800,992, 6,013,449, 6,020,135, 6,033,860, 6,040,138, 6,177,248 and 6,309,822. Genotyping and uses therefore are shown in U.S. Ser. Nos. 10/442,021, 10/013,598 (U.S. Patent Application Publication 20030036069), and U.S. Pat. Nos. 5,856,092, 6,300,063, 5,858,659, 6,284,460, 6,361,947, 6,368,799 and 6,333,179. Other uses are embodied in U.S. Pat. Nos. 5,871,928, 5,902,723, 6,045,996, 5,541,061, and 6,197,506.

The present invention also contemplates sample preparation methods in certain preferred embodiments. Prior to or concurrent with genotyping, the genomic sample may be amplified by a variety of mechanisms, some of which may employ PCR. See, e.g., PCR Technology: Principles and Applications for DNA Amplification (Ed. H. A. Erlich, Freeman Press, NY, N.Y., 1992); PCR Protocols: A Guide to Methods and Applications (Eds. Innis, et al., Academic Press, San Diego, Calif., 1990); Mattila et al., Nucleic Acids Res. 19, 4967 (1991); Eckert et al., PCR Methods and Applications 1, 17 (1991); PCR (Eds. McPherson et al., IRL Press, Oxford); and U.S. Pat. Nos. 4,683,202, 4,683,195, 4,800,159 4,965,188, and 5,333,675, and each of which is incorporated herein by reference in their entireties for all purposes. The sample may be amplified on the array. See, for example, U.S. Pat. No. 6,300,070 and U.S. Ser. No. 09/513,300, which are incorporated herein by reference. Other suitable amplification methods include the ligase chain reaction (LCR) (e.g., Wu and Wallace, Genomics 4, 560 (1989), Landegren et al., Science 241, 1077 (1988) and Barringer et al. Gene 89:117 (1990)), transcription amplification (Kwoh et al., Proc. Natl. Acad. Sci. USA 86, 1173 (1989) and WO88/10315), self-sustained sequence replication (Guatelli et al., Proc. Nat. Acad. Sci. USA, 87, 1874 (1990) and WO90/06995), selective amplification of target polynucleotide sequences (U.S. Pat. No. 6,410,276), consensus sequence primed polymerase chain reaction (CP-PCR) (U.S. Pat. No. 4,437,975), arbitrarily primed polymerase chain reaction (AP-PCR) (U.S. Pat. Nos. 5,413,909, 5,861,245) and nucleic acid based sequence amplification (NABSA). (See, U.S. Pat. Nos. 5,409,818, 5,554,517, and 6,063,603, each of which is incorporated herein by reference). Other amplification methods that may be used are described in, U.S. Pat. Nos. 5,242,794, 5,494,810, 4,988,617 and in U.S. Ser. No. 09/854,317, each of which is incorporated herein by reference.

Additional methods of sample preparation and techniques for reducing the complexity of a nucleic sample are described in Dong et al., Genome Research 11, 1418 (2001), in U.S. Pat. Nos. 6,361,947, 6,391,592 and U.S. Ser. Nos. 09/916,135, 09/920,491 (U.S. Patent Application Publication 20030096235), 09/910,292 (U.S. Patent Application Publication 20030082543), and 10/013,598.

Methods for conducting polynucleotide hybridization assays have been well developed in the art. Hybridization assay procedures and conditions will vary depending on the application and are selected in accordance with the general binding methods known including those referred to in: Maniatis et al. Molecular Cloning: A Laboratory Manual (2^{nd} Ed. Cold Spring Harbor, N.Y., 1989); Berger and Kimmel Methods in Enzymology, Vol. 152, Guide to Molecular Cloning Techniques (Academic Press, Inc., San Diego, Calif., 1987); Young and Davism, P.N.A.S, 80: 1194 (1983). Methods and apparatus for carrying out repeated and controlled hybridization reactions have been described in U.S. Pat. Nos. 5,871,928, 5,874,219, 6,045,996 and 6,386,749, 6,391,623 each of which are incorporated herein by reference

The present invention also contemplates signal detection of hybridization between ligands in certain preferred embodiments. See U.S. Pat. Nos. 5,143,854, 5,578,832; 5,631,734; 5,834,758; 5,936,324; 5,981,956; 6,025,601; 6,141,096; 6,185,030; 6,201,639; 6,218,803; and 6,225,625, in U.S. Ser. No. 10/389,194 and in PCT Application PCT/US99/06097 (published as WO99/47964), each of which also is hereby incorporated by reference in its entirety for all purposes.

Methods and apparatus for signal detection and processing of intensity data are disclosed in, for example, U.S. Pat. Nos. 5,143,854, 5,547,839, 5,578,832, 5,631,734, 5,800,992, 5,834,758; 5,856,092, 5,902,723, 5,936,324, 5,981,956, 6,025,601, 6,090,555, 6,141,096, 6,185,030, 6,201,639; 6,218,803; and 6,225,625, in U.S. Ser. Nos. 10/389,194, 10/913,102, 10/846,261, 11/260,617 and in PCT Application PCT/US99/06097 (published as WO99/47964), each of which also is hereby incorporated by reference in its entirety for all purposes.

The practice of the present invention may also employ conventional biology methods, software and systems. Computer software products of the invention typically include computer readable medium having computer-executable instructions for performing the logic steps of the method of the invention. Suitable computer readable medium include floppy disk, CD-ROM/DVD/DVD-ROM, hard-disk drive, flash memory, ROM/RAM, magnetic tapes and etc. The computer executable instructions may be written in a suitable computer language or combination of several languages. Basic computational biology methods are described in, e.g. Setubal and Meidanis et al., Introduction to Computational Biology Methods (PWS Publishing Company, Boston, 1997); Salzberg, Searles, Kasif, (Ed.), Computational Methods in Molecular Biology, (Elsevier, Amsterdam, 1998); Rashidi and Buehler, Bioinformatics Basics Application in Biological Science and Medicine (CRC Press, London, 2000) and Ouelette and Bzevanis Bioinformatics: A Practical Guide for Analysis of Gene and Proteins (Wiley & Sons, Inc., 2^{nd} ed., 2001). See U.S. Pat. No. 6,420,108. The present invention may also make use of various computer program products and software for a variety of purposes, such as probe design, management of data, analysis, and instrument operation. See, U.S. Pat. Nos. 5,593,839, 5,795,716, 5,733,729, 5,974,164, 6,066,454, 6,090,555, 6,185,561, 6,188,783, 6,223,127, 6,229,911 and 6,308,170.

Additionally, the present invention may have preferred embodiments that include methods for providing genetic information over networks such as the Internet as shown in U.S. Ser. Nos. 10/197,621, 10/063,559 (United States Publication No. 20020183936), 10/065,856, 10/065,868, 10/328,818, 10/328,872, 10/423,403, and 60/482,389.

### b) Definitions

The term “array” as used herein refers to an intentionally created collection of molecules which can be prepared either synthetically or biosynthetically. The molecules in the array can be identical or different from each other. The array can assume a variety of formats, e.g., libraries of soluble molecules; libraries of compounds tethered to resin beads, silica chips, or other solid supports.

The term “biomonomer” as used herein refers to a single unit of biopolymer, which can be linked with the same or other biomonomers to form a biopolymer (for example, a single amino acid or nucleotide with two linking groups one or both of which may have removable protecting groups) or a single unit which is not part of a biopolymer. Thus, for example, a nucleotide is a biomonomer within an oligonucleotide biopolymer, and an amino acid is a biomonomer within a protein or peptide biopolymer; avidin, biotin, antibodies, antibody fragments, etc., for example, are also biomonomers.

The term “biopolymer” or “biological polymer” as used herein is intended to mean repeating units of biological or chemical moieties. Representative biopolymers include, but are not limited to, nucleic acids, oligonucleotides, amino acids, proteins, peptides, hormones, oligosaccharides, lipids, glycolipids, lipopolysaccharides, phospholipids, synthetic analogues of the foregoing, including, but not limited to, inverted nucleotides, peptide nucleic acids, Meta-DNA, and combinations of the above.

The term “biopolymer synthesis” as used herein is intended to encompass the synthetic production, both organic and inorganic, of a biopolymer. Related to a bioploymer is a “biomonomer”.

The term “complementary” as used herein refers to the hybridization or base pairing between nucleotides or nucleic acids, such as, for instance, between the two strands of a double stranded DNA molecule or between an oligonucleotide primer and a primer binding site on a single stranded nucleic acid to be sequenced or amplified. Complementary nucleotides are, generally, A and T (or A and U), or C and G. Two single stranded RNA or DNA molecules are said to be complementary when the nucleotides of one strand, optimally aligned and compared and with appropriate nucleotide insertions or deletions, pair with at least about 80% of the nucleotides of the other strand, usually at least about 90% to 95%, and more preferably from about 98 to 100%. Alternatively, complementarity exists when an RNA or DNA strand will hybridize under selective hybridization conditions to its complement. Typically, selective hybridization will occur when there is at least about 65% complementary over a stretch of at least 14 to 25 nucleotides, preferably at least about 75%, more preferably at least about 90% complementary. See, M. Kanehisa Nucleic Acids Res. 12:203 (1984), incorporated herein by reference.

The term “combinatorial synthesis strategy” as used herein refers to a combinatorial synthesis strategy is an ordered strategy for parallel synthesis of diverse polymer sequences by sequential addition of reagents which may be represented by a reactant matrix and a switch matrix, the product of which is a product matrix. A reactant matrix is a l column by m row matrix of the building blocks to be added. The switch matrix is all or a subset of the binary numbers, preferably ordered, between l and m arranged in columns. A “binary strategy” is one in which at least two successive steps illuminate a portion, often half, of a region of interest on the substrate. In a binary synthesis strategy, all possible compounds which can be formed from an ordered set of reactants are formed. In most preferred embodiments, binary synthesis refers to a synthesis strategy which also factors a previous addition step. For example, a strategy in which a switch matrix for a masking strategy halves regions that were previously illuminated, illuminating about half of the previously illuminated region and protecting the remaining half (while also protecting about half of previously protected regions and illuminating about half of previously protected regions). It will be recognized that binary rounds may be interspersed with non-binary rounds and that only a portion of a substrate may be subjected to a binary scheme. A combinatorial “masking” strategy is a synthesis which uses light or other spatially selective deprotecting or activating agents to remove protecting groups from materials for addition of other materials such as amino acids.

The term “complex population or mixed population” as used herein refers to any sample containing both desired and undesired nucleic acids. As a non-limiting example, a complex population of nucleic acids may be total genomic DNA, total genomic RNA or a combination thereof. Moreover, a complex population of nucleic acids may have been enriched for a given population but include other undesirable populations. For example, a complex population of nucleic acids may be a sample which has been enriched for desired messenger RNA (mRNA) sequences but still includes some undesired ribosomal RNA sequences (rRNA).

The term “effective amount” as used herein refers to an amount sufficient to induce a desired result.

The term “genome” as used herein is all the genetic material in the chromosomes of an organism. DNA derived from the genetic material in the chromosomes of a particular organism is genomic DNA. A genomic library is a collection of clones made from a set of randomly generated overlapping DNA fragments representing the entire genome of an organism.

The term “hybridization conditions” as used herein will typically include salt concentrations of less than about 1M, more usually less than about 500 mM and preferably less than about 200 mM. Hybridization temperatures can be as low as 5.degree. C., but are typically greater than 22.degree. C., more typically greater than about 30.degree. C., and preferably in excess of about 37.degree. C. Longer fragments may require higher hybridization temperatures for specific hybridization. As other factors may affect the stringency of hybridization, including base composition and length of the complementary strands, presence of organic solvents and extent of base mismatching, the combination of parameters is more important than the absolute measure of any one alone.

The term “hybridization” as used herein refers to the process in which two single-stranded polynucleotides bind non-covalently to form a stable double-stranded polynucleotide; triple-stranded hybridization is also theoretically possible. The resulting (usually) double-stranded polynucleotide is a “hybrid.” The proportion of the population of polynucleotides that forms stable hybrids is referred to herein as the “degree of hybridization.” Hybridizations are usually performed under stringent conditions, for example, at a salt concentration of no more than 1 M and a temperature of at least 25° C. For example, conditions of 5×SSPE (750 mM NaCl, 50 mM NaPhosphate, 5 mM EDTA, pH 7.4) and a temperature of 25-30° C. are suitable for allele-specific probe hybridizations. For stringent conditions, see, for example, Sambrook, Fritsche and Maniatis. “Molecular Cloning A laboratory Manual” 2^{nd} Ed. Cold Spring Harbor Press (1989) which is hereby incorporated by reference in its entirety for all purposes above.

Hybridizations, e.g., allele-specific probe hybridizations, are generally performed under stringent conditions. For example, conditions where the salt concentration is no more than about 1 Molar (M) and a temperature of at least 25 degrees-Celsius (° C.), e.g., 750 mM NaCl, 50 mM NaPhosphate, 5 mM EDTA, pH 7.4 (5×SSPE) and a temperature of from about 25 to about 30° C.

The term “hybridization probes” as used herein are oligonucleotides capable of binding in a base-specific manner to a complementary strand of nucleic acid. Such probes include peptide nucleic acids, as described in Nielsen et al., Science 254, 1497-1500 (1991), and other nucleic acid analogs and nucleic acid mimetics.

The term “hybridizing specifically to” as used herein refers to the binding, duplexing, or hybridizing of a molecule only to a particular nucleotide sequence or sequences under stringent conditions when that sequence is present in a complex mixture (e.g., total cellular) DNA or RNA.

The term “initiation biomonomer” or “initiator biomonomer” as used herein is meant to indicate the first biomonomer which is covalently attached via reactive nucleophiles to the surface of the polymer, or the first biomonomer which is attached to a linker or spacer arm attached to the polymer, the linker or spacer arm being attached to the polymer via reactive nucleophiles.

The term “isolated nucleic acid” as used herein mean an object species invention that is the predominant species present (i.e., on a molar basis it is more abundant than any other individual species in the composition). Preferably, an isolated nucleic acid comprises at least about 50, 80 or 90% (on a molar basis) of all macromolecular species present. Most preferably, the object species is purified to essential homogeneity (contaminant species cannot be detected in the composition by conventional detection methods).

The term “ligand” as used herein refers to a molecule that is recognized by a particular receptor. The agent bound by or reacting with a receptor is called a “ligand,” a term which is definitionally meaningful only in terms of its counterpart receptor. The term “ligand” does not imply any particular molecular size or other structural or compositional feature other than that the substance in question is capable of binding or otherwise interacting with the receptor. Also, a ligand may serve either as the natural ligand to which the receptor binds, or as a functional analogue that may act as an agonist or antagonist. Examples of ligands that can be investigated by this invention include, but are not restricted to, agonists and antagonists for cell membrane receptors, toxins and venoms, viral epitopes, hormones (e.g., opiates, steroids, etc.), hormone receptors, peptides, enzymes, enzyme substrates, substrate analogs, transition state analogs, cofactors, drugs, proteins, and antibodies.

The term “linkage disequilibrium or allelic association” as used herein refers to the preferential association of a particular allele or genetic marker with a specific allele, or genetic marker at a nearby chromosomal location more frequently than expected by chance for any particular allele frequency in the population. For example, if locus X has alleles a and b, which occur equally frequently, and linked locus Y has alleles c and d, which occur equally frequently, one would expect the combination ac to occur with a frequency of 0.25. If ac occurs more frequently, then alleles a and c are in linkage disequilibrium. Linkage disequilibrium may result from natural selection of certain combination of alleles or because an allele has been introduced into a population too recently to have reached equilibrium with linked alleles.

The term “mixed population” as used herein refers to a complex population.

The term “monomer” as used herein refers to any member of the set of molecules that can be joined together to form an oligomer or polymer. The set of monomers useful in the present invention includes, but is not restricted to, for the example of (poly)peptide synthesis, the set of L-amino acids, D-amino acids, or synthetic amino acids. As used herein, “monomer” refers to any member of a basis set for synthesis of an oligomer. For example, dimers of L-amino acids form a basis set of 400 “monomers” for synthesis of polypeptides. Different basis sets of monomers may be used at successive steps in the synthesis of a polymer. The term “monomer” also refers to a chemical subunit that can be combined with a different chemical subunit to form a compound larger than either subunit alone.

The term “mRNA” or “mRNA transcripts” as used herein, include, but not limited to pre-mRNA transcript(s), transcript processing intermediates, mature mRNA(s) ready for translation and transcripts of the gene or genes, or nucleic acids derived from the mRNA transcript(s). Transcript processing may include splicing, editing and degradation. As used herein, a nucleic acid derived from an mRNA transcript refers to a nucleic acid for whose synthesis the mRNA transcript or a subsequence thereof has ultimately served as a template. Thus, a cDNA reverse transcribed from an mRNA, an RNA transcribed from that cDNA, a DNA amplified from the cDNA, an RNA transcribed from the amplified DNA, etc., are all derived from the mRNA transcript and detection of such derived products is indicative of the presence and/or abundance of the original transcript in a sample. Thus, mRNA derived samples include, but are not limited to, mRNA transcripts of the gene or genes, cDNA reverse transcribed from the mRNA, cRNA transcribed from the cDNA, DNA amplified from the genes, RNA transcribed from amplified DNA, and the like.

The term “nucleic acid library or array” as used herein refers to an intentionally created collection of nucleic acids which can be prepared either synthetically or biosynthetically and screened for biological activity in a variety of different formats (e.g., libraries of soluble molecules; and libraries of oligos tethered to resin beads, silica chips, or other solid supports). Additionally, the term “array” is meant to include those libraries of nucleic acids which can be prepared by spotting nucleic acids of essentially any length (e.g., from 1 to about 1000 nucleotide monomers in length) onto a substrate. The term “nucleic acid” as used herein refers to a polymeric form of nucleotides of any length, either ribonucleotides, deoxyribonucleotides or peptide nucleic acids (PNAs), that comprise purine and pyrimidine bases, or other natural, chemically or biochemically modified, non-natural, or derivatized nucleotide bases. The backbone of the polynucleotide can comprise sugars and phosphate groups, as may typically be found in RNA or DNA, or modified or substituted sugar or phosphate groups. A polynucleotide may comprise modified nucleotides, such as methylated nucleotides and nucleotide analogs. The sequence of nucleotides may be interrupted by non-nucleotide components. Thus the terms nucleoside, nucleotide, deoxynucleoside and deoxynucleotide generally include analogs such as those described herein. These analogs are those molecules having some structural features in common with a naturally occurring nucleoside or nucleotide such that when incorporated into a nucleic acid or oligonucleoside sequence, they allow hybridization with a naturally occurring nucleic acid sequence in solution. Typically, these analogs are derived from naturally occurring nucleosides and nucleotides by replacing and/or modifying the base, the ribose or the phosphodiester moiety. The changes can be tailor made to stabilize or destabilize hybrid formation or enhance the specificity of hybridization with a complementary nucleic acid sequence as desired.

The term “nucleic acids” as used herein may include any polymer or oligomer of pyrimidine and purine bases, preferably cytosine, thymine, and uracil, and adenine and guanine, respectively. See Albert L. Lehninger, PRINCIPLES OF BIOCHEMISTRY, at 793-800 (Worth Pub. 1982). Indeed, the present invention contemplates any deoxyribonucleotide, ribonucleotide or peptide nucleic acid component, and any chemical variants thereof, such as methylated, hydroxymethylated or glucosylated forms of these bases, and the like. The polymers or oligomers may be heterogeneous or homogeneous in composition, and may be isolated from naturally-occurring sources or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states.

The term “oligonucleotide” or “polynucleotide” as used herein refers to a nucleic acid ranging from at least 2, preferable at least 8, and more preferably at least 20 nucleotides in length or a compound that specifically hybridizes to a polynucleotide. Polynucleotides of the present invention include sequences of deoxyribonucleic acid (DNA) or ribonucleic acid (RNA) which may be isolated from natural sources, recombinantly produced or artificially synthesized and mimetics thereof. A further example of a polynucleotide of the present invention may be peptide nucleic acid (PNA). The invention also encompasses situations in which there is a nontraditional base pairing such as Hoogsteen base pairing which has been identified in certain tRNA molecules and postulated to exist in a triple helix. “Polynucleotide” and “oligonucleotide” are used interchangeably in this application.

The term “probe” as used herein refers to a surface-immobilized molecule that can be recognized by a particular target. See U.S. Pat. No. 6,582,908 for an example of arrays having all possible combinations of probes with 10, 12, and more bases. Examples of probes that can be investigated by this invention include, but are not restricted to, agonists and antagonists for cell membrane receptors, toxins and venoms, viral epitopes, hormones (e.g., opioid peptides, steroids, etc.), hormone receptors, peptides, enzymes, enzyme substrates, cofactors, drugs, lectins, sugars, oligonucleotides, nucleic acids, oligosaccharides, proteins, and monoclonal antibodies.

The term “primer” as used herein refers to a single-stranded oligonucleotide capable of acting as a point of initiation for template-directed DNA synthesis under suitable conditions e.g., buffer and temperature, in the presence of four different nucleoside triphosphates and an agent for polymerization, such as, for example, DNA or RNA polymerase or reverse transcriptase. The length of the primer, in any given case, depends on, for example, the intended use of the primer, and generally ranges from 15 to 30 nucleotides. Short primer molecules generally require cooler temperatures to form sufficiently stable hybrid complexes with the template. A primer need not reflect the exact sequence of the template but must be sufficiently complementary to hybridize with such template. The primer site is the area of the template to which a primer hybridizes. The primer pair is a set of primers including a 5′ upstream primer that hybridizes with the 5′ end of the sequence to be amplified and a 3′ downstream primer that hybridizes with the complement of the 3′ end of the sequence to be amplified.

The term “polymorphism” as used herein refers to the occurrence of two or more genetically determined alternative sequences or alleles in a population. A polymorphic marker or site is the locus at which divergence occurs. Preferred markers have at least two alleles, each occurring at frequency of greater than 1%, and more preferably greater than 10% or 20% of a selected population. A polymorphism may comprise one or more base changes, an insertion, a repeat, or a deletion. A polymorphic locus may be as small as one base pair. Polymorphic markers include restriction fragment length polymorphisms, variable number of tandem repeats (VNTR's), hypervariable regions, minisatellites, dinucleotide repeats, trinucleotide repeats, tetranucleotide repeats, simple sequence repeats, and insertion elements such as Alu. The first identified allelic form is arbitrarily designated as the reference form and other allelic forms are designated as alternative or variant alleles. The allelic form occurring most frequently in a selected population is sometimes referred to as the wildtype form. Diploid organisms may be homozygous or heterozygous for allelic forms. A diallelic polymorphism has two forms. A triallelic polymorphism has three forms. Single nucleotide polymorphisms (SNPs) are included in polymorphisms.

The term “receptor” as used herein refers to a molecule that has an affinity for a given ligand. Receptors may be naturally-occurring or manmade molecules. Also, they can be employed in their unaltered state or as aggregates with other species. Receptors may be attached, covalently or noncovalently, to a binding member, either directly or via a specific binding substance. Examples of receptors which can be employed by this invention include, but are not restricted to, antibodies, cell membrane receptors, monoclonal antibodies and antisera reactive with specific antigenic determinants (such as on viruses, cells or other materials), drugs, polynucleotides, nucleic acids, peptides, cofactors, lectins, sugars, polysaccharides, cells, cellular membranes, and organelles. Receptors are sometimes referred to in the art as anti-ligands. As the term receptors is used herein, no difference in meaning is intended. A “Ligand Receptor Pair” is formed when two macromolecules have combined through molecular recognition to form a complex. Other examples of receptors which can be investigated by this invention include but are not restricted to those molecules shown in U.S. Pat. No. 5,143,854, which is hereby incorporated by reference in its entirety.

The term “solid support”, “support”, and “substrate” as used herein are used interchangeably and refer to a material or group of materials having a rigid or semi-rigid surface or surfaces. In many embodiments, at least one surface of the solid support will be substantially flat, although in some embodiments it may be desirable to physically separate synthesis regions for different compounds with, for example, wells, raised regions, pins, etched trenches, or the like. According to other embodiments, the solid support(s) will take the form of beads, resins, gels, microspheres, or other geometric configurations. See U.S. Pat. No. 5,744,305 for exemplary substrates.

The term “target” as used herein refers to a molecule that has an affinity for a given probe. Targets may be naturally-occurring or man-made molecules. Also, they can be employed in their unaltered state or as aggregates with other species. Targets may be attached, covalently or noncovalently, to a binding member, either directly or via a specific binding substance. Examples of targets which can be employed by this invention include, but are not restricted to, antibodies, cell membrane receptors, monoclonal antibodies and antisera reactive with specific antigenic determinants (such as on viruses, cells or other materials), drugs, oligonucleotides, nucleic acids, peptides, cofactors, lectins, sugars, polysaccharides, cells, cellular membranes, and organelles. Targets are sometimes referred to in the art as anti-probes. As the term targets is used herein, no difference in meaning is intended. A “Probe Target Pair” is formed when two macromolecules have combined through molecular recognition to form a complex.

### c) Embodiments of the Present Invention

Embodiments of consumable elements for use with fluid processing and scanning systems are described herein that are enabled to process and acquires images comprising features of a probe array that may include feature sizes in a range of 24 μm, 5 μm, 1 μm or smaller in a dimension (such as the side of a square, side of a rectangle, or diameter of a spot). Efficient processing is performed in the presently described embodiments by consumable elements and instrumentation enabled to provide user 101 with “walk-away” freedom virtually eliminating the need for intervention between processing steps, and conservation of reagent usage to reduction of experimental costs.

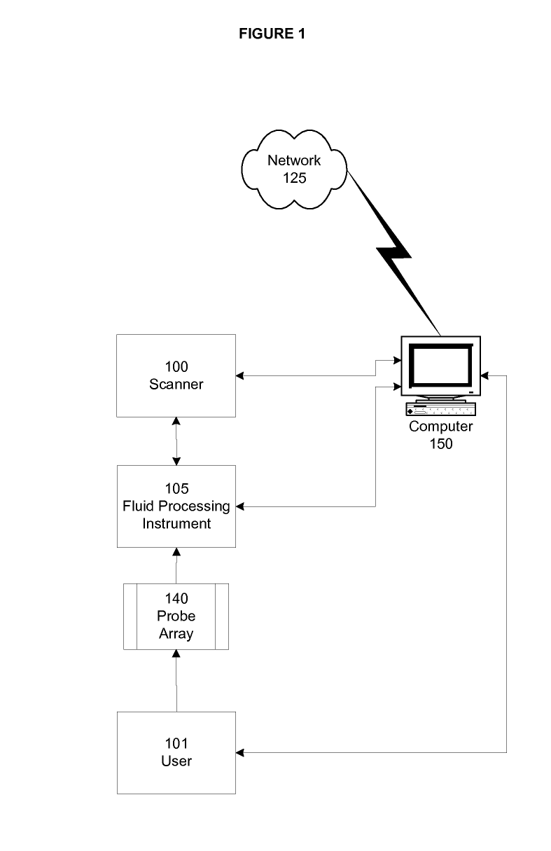

Probe Array 140: An illustrative example of probe array 140 is provided in FIGS. 1, 2, and 3. Descriptions of probe arrays are provided above with respect to “Nucleic Acid Probe arrays” and other related disclosure. In various implementations, probe array 140 may be disposed in a cartridge or housing such as, for example, the GeneChip® probe array available from Affymetrix, Inc. of Santa Clara Calif. Examples of probe arrays and associated cartridges or housings may be found in U.S. Pat. Nos. 5,945,334, 6,287,850, 6,399,365, 6,551,817, each of which is also hereby incorporated by reference herein in its entirety for all purposes. In addition, some embodiments of probe array 140 may be associated with pegs or posts, where for instance probe array 140 may be affixed via gluing, welding, or other means known in the related art to the peg or post that may be operatively coupled to a tray, strip or other type of similar substrate. Examples with embodiments of probe array 140 associated with pegs or posts may be found in U.S. patent Ser. No. 10/826,577, titled “Immersion Array Plates for Interchangeable Microtiter Well Plates”, filed Apr. 16, 2004, which is hereby incorporated by reference herein in its entirety for all purposes.

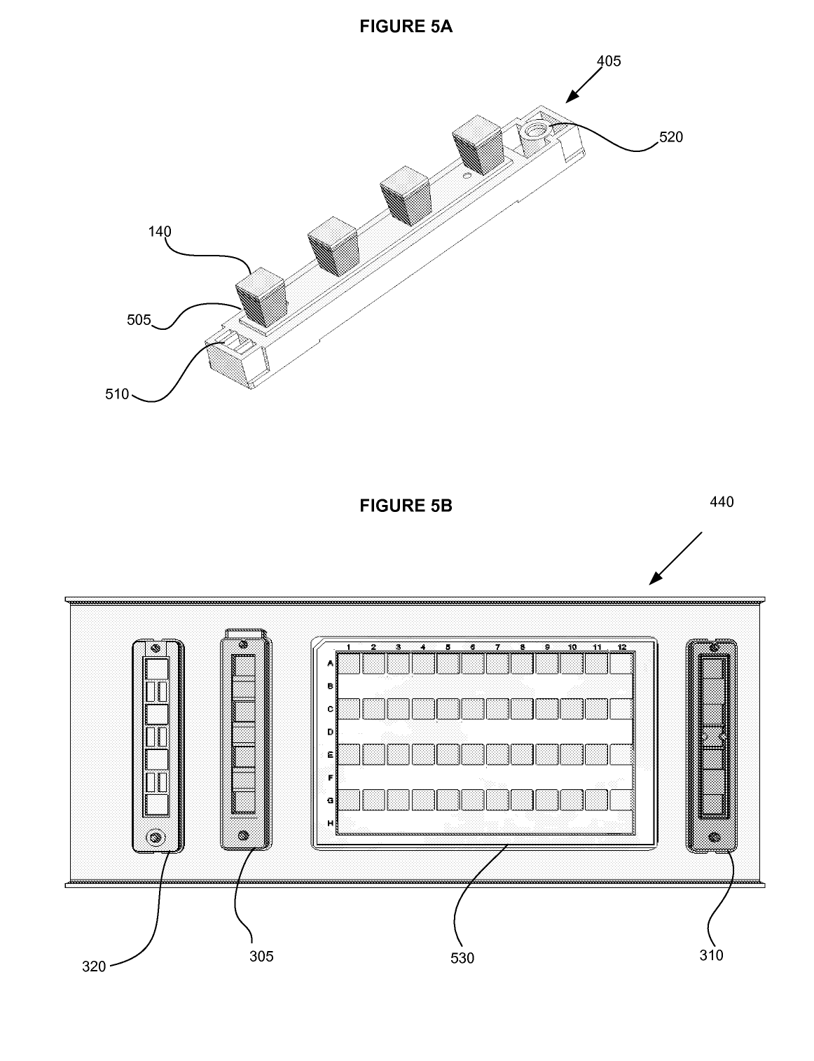

For example, FIG. 5A illustrates an implementation of a 4 peg embodiment that comprises 4 implementations of probe array 140 disposed upon peg 505 that substantially separates probe array 140 from strip 405. In the present example, each embodiment of probe array 140/peg 505 associated with 4 peg format may include an 8 mm square and may be spaced at a 9 mm pitch, 18 mm pitch, or other spacing. Those of ordinary skill in the related art will appreciate that the representations provided in FIG. 5A are for the purposes of illustration only, and that the numbers of probe array 140/peg 505 implementations associated with a particular substrate could vary greatly by embodiment including a single probe array/peg embodiment.

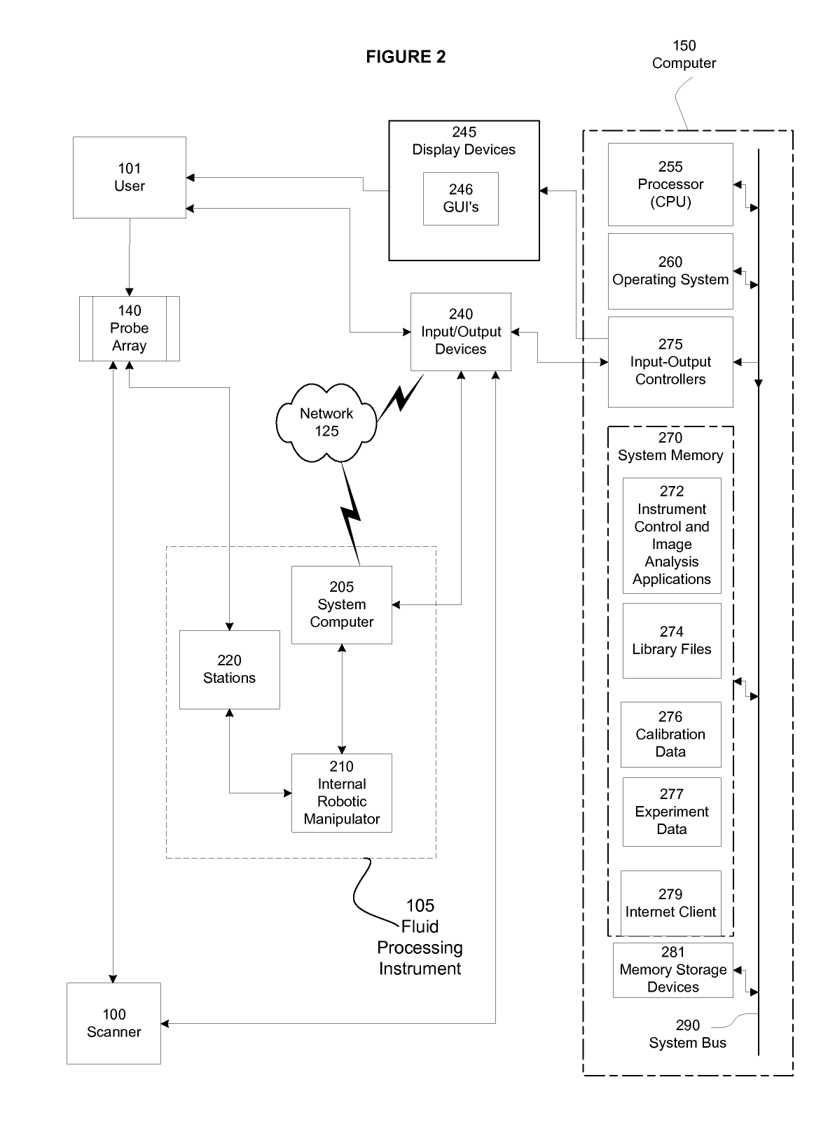

Computer 150: An illustrative example of computer 150 is provided in FIG. 1 and also in greater detail in FIG. 2. Computer 150 may be any type of computer platform such as a workstation, a personal computer, a server, or any other present or future computer. Computer 150 typically includes known components such as a processor 255, an operating system 260, system memory 270, memory storage devices 281, and input-output controllers 275, input-output devices 240, and display devices 245. Display devices 245 may include display devices that provides visual information, this information typically may be logically and/or physically organized as an array of pixels. A Graphical user interface (GUI) controller may also be included that may comprise any of a variety of known or future software programs for providing graphical input and output interfaces such as for instance GUI's 246. For example, GUI's 246 may provide one or more graphical representations to a user, such as user 101, and also be enabled to process user inputs via GUI's 246 using means of selection or input known to those of ordinary skill in the related art.

It will be understood by those of ordinary skill in the relevant art that there are many possible configurations of the components of computer 150 and that some components that may typically be included in computer 150 are not shown, such as cache memory, a data backup unit, and many other devices. Processor 255 may be a commercially available processor such as an Itanium® or Pentium® processor made by Intel Corporation, a SPARC® processor made by Sun Microsystems, an Athalon™ or Opteron™ processor made by AMD corporation, or it may be one of other processors that are or will become available. Some embodiments of processor 255 may also include what are referred to as Multi-core processors and/or be enabled to employ parallel processing technology in a single or multi-core configuration. For example, a multi-core architecture typically comprises two or more processor “execution cores”. In the present example each execution core may perform as an independent processor that enables parallel execution of multiple threads. In addition, those of ordinary skill in the related will appreciate that processor 255 may be configured in what is generally referred to as 32 or 64 bit architectures, or other architectural configurations now known or that may be developed in the future.

Processor 255 executes operating system 260, which may be, for example, a Windows®-type operating system (such as Windows® XP) from the Microsoft Corporation; the Mac OS X operating system from Apple Computer Corp. (such as 7.5 Mac OS X v10.4 “Tiger” or 7.6 Mac OS X v10.5 “Leopard” operating systems); a Unix® or Linux-type operating system available from many vendors or what is referred to as an open source; another or a future operating system; or some combination thereof. Operating system 260 interfaces with firmware and hardware in a well-known manner, and facilitates processor 255 in coordinating and executing the functions of various computer programs that may be written in a variety of programming languages. Operating system 260, typically in cooperation with processor 255, coordinates and executes functions of the other components of computer 150. Operating system 260 also provides scheduling, input-output control, file and data management, memory management, and communication control and related services, all in accordance with known techniques.

System memory 270 may be any of a variety of known or future memory storage devices. Examples include any commonly available random access memory (RAM), magnetic medium such as a resident hard disk or tape, an optical medium such as a read and write compact disc, or other memory storage device. Memory storage devices 281 may be any of a variety of known or future devices, including a compact disk drive, a tape drive, a removable hard disk drive, USB or flash drive, or a diskette drive. Such types of memory storage devices 281 typically read from, and/or write to, a program storage medium (not shown) such as, respectively, a compact disk, magnetic tape, removable hard disk, USB or flash drive, or floppy diskette. Any of these program storage media, or others now in use or that may later be developed, may be considered a computer program product. As will be appreciated, these program storage media typically store a computer software program and/or data. Computer software programs, also called computer control logic, typically are stored in system memory 270 and/or the program storage device used in conjunction with memory storage device 281.

In some embodiments, a computer program product is described comprising a computer usable medium having control logic (computer software program, including program code) stored therein. The control logic, when executed by processor 255, causes processor 255 to perform functions described herein. In other embodiments, some functions are implemented primarily in hardware using, for example, a hardware state machine. Implementation of the hardware state machine so as to perform the functions described herein will be apparent to those skilled in the relevant arts.

Input-output controllers 275 could include any of a variety of known devices for accepting and processing information from a user, whether a human or a machine, whether local or remote. Such devices include, for example, modern cards, wireless cards, network interface cards, sound cards, or other types of controllers for any of a variety of known input devices. Output controllers of input-output controllers 275 could include controllers for any of a variety of known display devices for presenting information to a user, whether a human or a machine, whether local or remote. In the illustrated embodiment, the functional elements of computer 150 communicate with each other via system bus 290. Some of these communications may be accomplished in alternative embodiments using network or other types of remote communications.

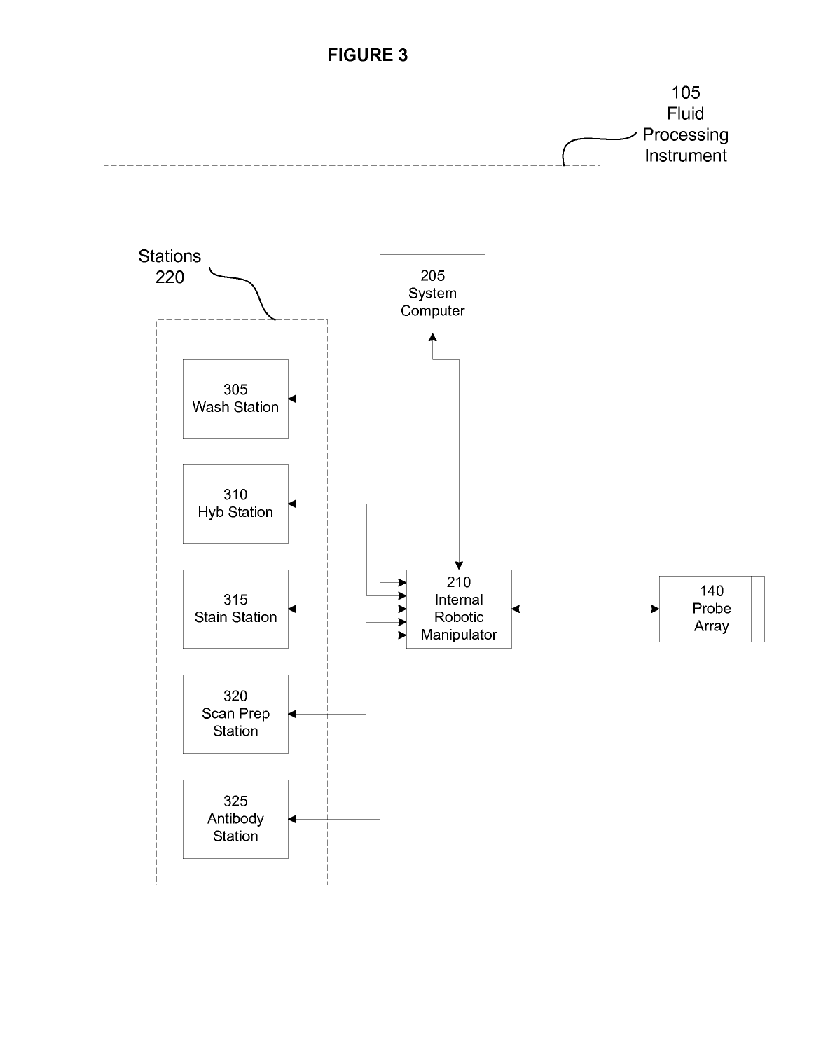

As will be evident to those skilled in the relevant art, an instrument control and image processing application, such as for instance an implementation of instrument control and image processing applications 272 illustrated in FIG. 3, if implemented in software, may be loaded into and executed from system memory 270 and/or memory storage device 281. All or portions of the instrument control and image processing applications may also reside in a read-only memory or similar device of memory storage device 281, such devices not requiring that the instrument control and image processing applications first be loaded through input-output controllers 275. It will be understood by those skilled in the relevant art that the instrument control and image processing applications, or portions of it, may be loaded by processor 255 in a known manner into system memory 270, or cache memory (not shown), or both, as advantageous for execution. Also illustrated in FIG. 2 are library files 274, calibration data 276, experiment data 277, and internet client 279 stored in system memory 270. For example, experiment data 277 could include data related to one or more experiments or assays such as excitation wavelength ranges, emission wavelength ranges, extinction coefficients and/or associated excitation power level values, or other values associated with one or more fluorescent labels. Additionally, internet client 279 may include an application enabled to accesses a remote service on another computer using a network that may for instance comprise what are generally referred to as “Web Browsers”. In the present example some commonly employed web browsers include Netscape® 8.0 available from Netscape Communications Corp., Microsoft® Internet Explorer 6 with SPI available from Microsoft Corporation, Mozilla Firefox® 1.5 from the Mozilla Corporation, Safari 2.0 from Apple Computer Corp., or other type of web browser currently known in the art or to be developed in the future. Also, in the same or other embodiments internet client 279 may include, or could be an element of, specialized software applications enabled to access remote information via a network such as network 125 such as, for instance, the GeneChip® Data Analysis Software (GDAS) package or Chromosome Copy Number Tool (CNAT) both available from Affymetrix, Inc. of Santa Clara Calif. that are each enabled to access information from remote sources, and in particular probe array annotation information from the NetAffx™ web site hosted on one or more servers provided by Affymetrix, Inc.

Network 125 may include one or more of the many various types of networks well known to those of ordinary skill in the art. For example, network 125 may include a local or wide area network that employs what is commonly referred to as a TCP/IP protocol suite to communicate, that may include a network comprising a worldwide system of interconnected computer networks that is commonly referred to as the internet, or could also include various intranet architectures. Those of ordinary skill in the related arts will also appreciate that some users in networked environments may prefer to employ what are generally referred to as “firewalls” (also sometimes referred to as Packet Filters, or Border Protection Devices) to control information traffic to and from hardware and/or software systems. For example, firewalls may comprise hardware or software elements or some combination thereof and are typically designed to enforce security policies put in place by users, such as for instance network administrators, etc.

Instrument control and image processing applications 272: Instrument control and image processing applications 272 may comprise any of a variety of known or future image processing applications. Some examples of known instrument control and image processing applications include the Affymetrix® Microarray Suite, and Affymetrix® GeneChip® Operating Software (hereafter referred to as GCOS) applications. Typically, embodiments of applications 272 may be loaded into system memory 370 and/or memory storage device 381.

Those of ordinary skill in the related art will appreciate that applications 272 may be stored for execution on any compatible computer system, such as computer 150. For example, the described embodiments of applications 272 may, for example, include the Affymetrix® command-console™ software. Embodiments of applications 272 may advantageously provide what is referred to as a modular interface for one or more computers or workstations and one or more servers, as well as one or more instruments. The term “modular” as used herein generally refers to elements that may be integrated to and interact with a core element in order to provide a flexible, updateable, and customizable platform. For example, as will be described in greater detail below applications 272 may comprise a “core” software element enabled to communicate and perform primary functions necessary for any instrument control and image processing application. Such primary functionality may include communication over various network architectures, or data processing functions such as processing raw intensity data into a .dat file. In the present example, modular software elements, such as for instance what may be referred to as a plug-in module, may be interfaced with the core software element to perform more specific or secondary functions, such as for instance functions that are specific to particular instruments. In particular, the specific or secondary functions may include functions customizable for particular applications desired by user 101. Further, integrated modules and the core software element are considered to be a single software application, and referred to as applications 272.

In the presently described implementation, applications 272 may communicate with, and receive instruction or information from, or control one or more elements or processes of one or more servers, one or more workstations, and one or more instruments. Also, embodiments of server 120 or computer 150 with an implementation of applications 272 stored thereon could be located locally or remotely and communicate with one or more additional servers and/or one or more other computers/workstations or instruments.

In some embodiments, applications 272 may be capable of data encryption/decryption functionality. For example, it may be desirable to encrypt data, files, information associated with GUI's 246, or other information that may be transferred over network 125 to one or more remote computers or servers for data security and confidentiality purposes. For example, some embodiments of probe array 140 may be employed for diagnostic purposes where the data may be associated with a patient and/or a diagnosis of a disease or medical condition. It is desirable in many applications to protect the data using encryption for confidentiality of patient information. In addition, one-way encryption technologies may be employed in situations where access should be limited to only selected parties such as a patient and their physician. In the present example, only the selected parties have the key to decrypt or associate the data with the patient. In some applications, the one-way encrypted data may be stored in one or more public databases or repositories where even the curator of the database or repository would be unable to associate the data with the user or otherwise decrypt the information. The described encryption functionality may also have utility in clinical trial applications where it may be desirable to isolate one or more data elements from each other for the purpose of confidentiality and/or removal of experimental biases.

Various embodiments of applications 272 may provide one or more interactive graphical user interfaces that allows user 101 to make selections based upon information presented in an embodiment of GUI 246. Those of ordinary skill will recognize that embodiments of GUI 246 may be coded in various language formats such as an HTML, XHTML, XML, javascript, Jscript, or other language known to those of ordinary skill in the art used for the creation or enhancement of “Web Pages” viewable and compatible with internet client 279. For example, internet client 279 may include various internet browsers such as Microsoft Internet Explorer, Netscape Navigator, Mozilla Firefox, Apple Safari, or other browsers known in the art. Applications of GUI's 246 viewable via one or more browsers may allow user 101 complete remote access to data, management, and registration functions without any other specialized software elements. Applications 272 may provide one or more implementations of interactive GUI's 246 that allow user 101 to select from a variety of options including data selection, experiment parameters, calibration values, and probe array information within the access to data, management, and registration functions.

In some embodiments, applications 272 may be capable of running on operating systems in a non-English format, where applications 272 can accept input from user 101 in various non-English language formats such as Chinese, French, Spanish etc., and output information to user 101 in the same or other desired language output. For example, applications 272 may present information to user 101 in various implementations of GUI 246 in a language output desired by user 101, and similarly receive input from user 101 in the desired language. In the present example, applications 272 is internationalized such that it is capable of interpreting the input from user 101 in the desired language where the input is acceptable input with respect to the functions and capabilities of applications 272.

Embodiments of applications 272 also include instrument control features, where the control functions of individual types or specific instruments such as scanner 100, an autoloader, or fluid handling system may be organized as plug-in type modules to applications 272. For example, each plug-in module may be a separate component and may provide definition of the instrument control features to applications 272. As described above, each plug-in module is functionally integrated with applications 272 when stored in system memory 270 and thus reference to applications 272 includes any integrated plug-in modules. In the present example, each instrument may have one or more associated embodiments of plug-in module that for instance may be specific to model of instrument, revision of instrument firmware or scripts, number and/or configuration of instrument embodiment, etc. Further, multiple embodiments of plug-in module for the same instrument such as scanner 100 may be stored in system memory 270 for use by applications 272, where user 101 may select the desired embodiment of module to employ, or alternatively such a selection of module may be defined by data encoded directly in a machine readable identifier or indirectly via the array file, library files, experiments files and so on.

The instrument control features may include the control of one or more elements of one or more instruments that could, for instance, include elements of a hybridization device, fluid processing instrument 105, autoloader, or scanner 100. The instrument control features may also be capable of receiving information from the one more instruments that could include experiment or instrument status, process steps, or other relevant information. The instrument control features could, for example, be under the control of or an element of the interface of applications 272. In some embodiments, a user may input desired control commands and/or receive the instrument control information via one of GUI's 246. Additional examples of instrument control via a GUI or other interface is provided in U.S. patent application Ser. No. 10/764,663, titled “System, Method and Computer Software Product for Instrument Control, Data Acquisition, Analysis, Management and Storage”, filed Jan. 26, 2004, which is hereby incorporated by reference herein in its entirety for all purposes.

In some embodiments, applications 272 may employ what may referred to as an “array file” that comprises data employed for various instruments, processing functions of images by applications 272, or other relevant information. Generally it is desirable to consolidate elements of data or metadata related to an embodiment of probe array 140, experiment, user, or some combination thereof, to a single file that is not duplicated (i.e. as embodiments of .dat file may be in certain applications), where duplication may sometimes be a source of error. The term “metadata” as used herein generally refers to data about data. It may also be desirable in some embodiments to restrict or prohibit the ability to overwrite data in the array file. Preferentially, new information may be appended to the array file rather than deleting or overwriting information, providing the benefit of traceability and data integrity (i.e. as may be required by some regulatory agencies). For example, an array file may be associated with one or more implementations of an embodiment of probe array 140, where the array file acts to unify data across a set of probe arrays 140. The array file may be created by applications 272 via a registration process, where user 101 inputs data into applications 272 via one or more of GUI's 246. In the present example, the array file may be associated by user 101 with a custom identifier that could include a machine readable identifier such as the machine readable identifiers described in greater detail below. Alternatively, applications 272 may create an array file and automatically associate the array file with a machine readable identifier that identifies an embodiment of probe array 140 (i.e. relationship between the machine readable identifier and probe array 140 may be assigned by a manufacturer). Applications 272 may employ various data elements for the creation or update of the array file from one or more library files, such as library files 274 or other library files.

Alternatively, the array file may comprise pointers to one or more additional data files comprising data related to an associated embodiment of probe array 140. For example, the manufacturer of probe array 140 or other user may provide library files 274 or other files that define characteristics such as probe identity; dimension and positional location (i.e. with respect to some fiducial reference or coordinate system) of the active area of probe array 140; various experimental parameters; instrument control parameters; or other types of useful information. In addition, the array file may also contain one or more metadata elements that could include one or more of a unique identifier for the array file, human readable form of a machine readable identifier, or other metadata elements. In addition, applications 272 may store data (i.e. as metadata, or stored data) that includes sample identifiers, array names, user parameters, event logs that may for instance include a value identifying the number of times an array has been scanned, relationship histories such as for instance the relationship between each .cel file and the one or more .dat files that were employed to generate the .cel file, and other types of data useful in for processing and data management.

For example, user 101 and/or automated data input devices or programs (not shown) may provide data related to the design or conduct of experiments. User 101 may specify an Affymetrix catalogue or custom chip type (e.g., Human Genome U133 plus 2.0 chip) either by selecting from a predetermined list presented in one or more of GUI's 246 or by scanning a bar code, Radio Frequency Identification (RFID), magnetic strip, or other means of electronic identification related to probe array 140 to read its type, part no., array identifier, etc. Applications 272 may associate the chip type, part no., array identifier with various scanning parameters stored in data tables or library files, such as library files 274 of computer 150, including the area of probe array 140 that is to be scanned, the location of chrome elements or other features on probe array 140 used for auto-focusing, the wavelength or intensity/power of excitation light to be used in reading the chip, and so on. Also, some embodiments of applications 272 may encode array files in a binary type format that may minimize the possibility of data corruption. However, applications 272 may be further enabled to export an array file in a number of different formats.

Also continuing the example above, some embodiments of RFID tags associated with embodiments of probe array 140 may be capable of “data logging” functionality where, for instance, each RFID tag or label may actively measure and record parameters of interest. In the present example, such parameters of interest may include environmental conditions such as temperature and/or humidity that the implementation of probe array 140 may have been exposed to. In the present example, user 101 may be interested in the environmental conditions because the biological integrity of some embodiments of probe array 140 may be affected by exposure to fluctuations of the environment. In some embodiments, applications 272 may extract the recorded environmental information from the RFID tag or label and store it in the array file, or some other file that has a pointer to or from the array file. In the same or alternative embodiments, applications 2721 may monitor the environmental conditions exposed to the probe array in real time, where applications 272 may regularly monitor information provided by one or more RFID tags simultaneously. Applications 272 may further analyze and employ such information for quality control purposes, for data normalization, or other purposes known in the related art. Some examples of RFID embodiments capable to recording environmental parameters include the ThermAssureRF™ RFID sensor available from Evidencia LLP of Memphis Tenn., or the Tempsens™ RFID datalogging label available from Exago Pty Ltd. of Australia.

Also, in the same or alternative embodiments, applications 272 may generate or access what may be referred to as a “plate” file. The plate file may encode one or more data elements such as pointers to one or more array files, and preferably may include pointers to a plurality of array files.

In some embodiments, raw image data is acquired from scanner 100 and operated upon by applications 272 to generate intermediate results. For example, raw intensity data acquired from scanner 100 may be directed to a .dat file generator and written to data files (*.dat) that comprises an intensity value for each pixel of data acquired from a scan of an embodiment of probe array 140. In the same or alternative embodiments it may be advantageous to scan sub areas (that may be referred to as sub arrays) of probe array 140 where the detected signal for each sub area scanned may be written to an individual embodiment of a .dat file. Continuing with the present example, applications 272 may also encode a unique identifier for each .dat file as well as a pointer to an associated embodiment of an array file as metadata into each .dat file generated. The term “pointer” as used herein generally refers to a programming language datatype, variable, or data object that references another data object, datatype, variable, etc. using a memory address or identifier of the referenced element in a memory storage device such as in system memory 270. In some embodiments the pointers comprise the unique identifiers of the files that are the subject of the pointing, such as for instance the pointer in a .dat file comprises the unique identifier of the array file. Additional examples of the generation and image processing of sub arrays is described in U.S. patent application Ser. No. 11/289,975, titled “System, Method, and Product for Analyzing Images Comprising Small Feature Sizes”, filed Nov. 30, 2005, which is hereby incorporated by reference herein in its entirety for all purpose.

Also, applications 272 may also include a .cel file generator that may produce one or more .cel files (*.cel) by processing each .dat file. Alternatively, some embodiments of .cel file generator may produce a single .cel file from processing multiple .dat files such as with the example of processing multiple sub-arrays described above. Similar to the .dat file described above each embodiment of .cel file may also include one or more metadata elements. For example, applications 272 may encode a unique identifier for each .cel file as well as a pointer to an associated array file and/or the one or more .dat files used to produce the .cel file.

Each .cel file contains, for each probe feature scanned by scanner 100, a single value representative of the intensities of pixels measured by scanner 100 for that probe. For example, this value may include a measure of the abundance of tagged mRNA's present in the target that hybridized to the corresponding probe. Many such mRNA's may be present in each probe, as a probe on a GeneChip® probe array may include, for example, millions of oligonucleotides designed to detect the mRNA's. Alternatively, the value may include a measure related to the sequence composition of DNA or other nucleic acid detected by the probes of a GeneChip® probe array. As described above, applications 272 receives image data derived from probe array 140 using scanner 100 and generates a .dat file that is then processed by applications 272 to produce a .cel intensity file, where applications 272 may utilize information from an array file in the image processing function. For instance, the .cel file generator may perform what is referred to as grid placement methods on the image data in each .dat file using data elements such as dimension information to determine and define the positional location of probe features in the image. Typically, the .cel file generator associates what may be referred to as a grid with the image data in a .dat file for the purpose of determining the positional relationship of probe features in the image with the known positions and identities of the probe features. The accurate registration of the grid with the image is important for the accuracy of the information in the resulting .cel file. Also, some embodiments of .cel file generator may provide user 101 with a graphical representation of a grid aligned to image data from a selected .dat file in an implementation of GUI 246, and further enable user 101 to manually refine the position of the grid placement using methods commonly employed such as placing a cursor over the grid, selecting such as by holding down a button on a mouse, and dragging the grid to a preferred positional relationship with the image. Applications 272 may then perform methods sometimes referred to as “feature extraction” to assign a value of intensity for each probe represented in the image as an area defined by the boundary lines of the grid. Examples of grid registration, methods of positional refinement, and feature extraction are described in U.S. Pat. Nos. 6,090,555; 6,611,767; 6,829,376, and U.S. patent application Ser. Nos. 10/391,882, and 10/197,369, each of which is hereby incorporated by reference herein in it's entirety for all purposes.

As noted, another file that may be generated by applications 272 is a .chp file using a .chp file generator. For example, each .chp file is derived from analysis of a .cel file combined in some cases with information derived from an array file, other lab data and/or library files 274 that specify details regarding the sequences and locations of probes and controls. In some embodiments, a machine readable identifier associated with probe array 140 may indicate the library file directly or indirectly via one or more identifiers in the array file, to employ for identification of the probes and their positional locations. The resulting data stored in the .chp file includes degrees of hybridization, absolute and/or differential (over two or more experiments) expression, genotype comparisons, detection of polymorphisms and mutations, and other analytical results.

In some alternative embodiments, user 101 may prefer to employ different applications to process data such as an independent analysis application. Embodiments of an analysis application may comprise any of a variety of known or probe array analysis applications, and particularly analysis applications specialized for use with embodiments of probe array 140 designed for genotyping or expression applications. Various embodiments of analysis application may exist such as applications developed by the probe array manufacturer for specialized embodiments of probe array 140, commercial third party software applications, open source applications, or other applications known in the art for specific analysis of data from probe arrays 140. Some examples of known genotyping analysis applications include the Affymetrix® GeneChip® Data Analysis System (GDAS), Affymetrix® GeneChip® Genotyping Analysis Software (GTYPE), Affymetrix® GeneChip® Targeted Genotyping Analysis Software (GTGS), and Affymetrix® GeneChip® Sequence Analysis Software (GSEQ) applications. Additional examples of genotyping analysis applications may be found in U.S. patent application Ser. Nos. 10/657,481; 10/986,963; and 11/157,768; each of which is hereby incorporated by reference herein in it's entirety for all purposes. Typically, embodiments of analysis applications may be loaded into system memory 270 and/or memory storage device 281 through one of input-output devices 240.

Some embodiments of analysis applications include executable code being stored in system memory 270. Applications 272 may be enabled to export .cel files, .dat files, or other files to an analysis application or enable access to such files on computer 150 by the analysis application. Import and/or export functionality for compatibility with specific systems or applications may be enabled by one or more integrated modules as described above with respect to plug-in modules. For example, an analysis application may be capable of performing specialized analysis of processed intensity data, such as the data in a .cel file. In the present example, user 101 may desire to process data associated with a plurality of implementations of probe array 140 and therefore the analysis application would receive a .cel file associated with each probe array for processing. In the present example, applications 272 forwards the appropriate files in response to queries or requests from the analysis application.

In the same or alternative examples, user 101 and/or the third party developers may employ what are referred to as software development kits that enable programmatic access into file formats, or the structure of applications 272. Therefore, developers of other software applications such as the described analysis application may integrate with and seamlessly add functionally to or utilize data from applications 272 that provides user 101 with a wide range of application and processing capability. Additional examples of software development kits associated with software or data related to probe arrays are described in U.S. Pat. No. 6,954,699, and U.S. application Ser. Nos. 10/764,663 and 11/215,900, each of which is hereby incorporated by reference herein in its entirety for all purposes.

Additional examples of .cel and .chp files are described with respect to the Affymetrix GeneChip® Operating Software or Affymetrix® Microarray Suite (as described, for example, in U.S. patent application Ser. Nos. 10/219,882, and 10/764,663, both of which are hereby incorporated herein by reference in their entireties for all purposes). For convenience, the term “file” often is used herein to refer to data generated or used by applications 272 and executable counterparts of other applications such as analysis application 380, where the data is written according a format such as the described .dat, .cel, and .chp formats. Further, the data files may also be used as input for applications 272 or other software capable of reading the format of the file.

Those of ordinary skill in the related art will appreciate that one or more operations of applications 272 may be performed by software or firmware associated with various instruments. For example, scanner 100 could include a computer that may include a firmware component that performs or controls one or more operations associated with scanner 100.

Yet another example of instrument control and image analysis applications is described in U.S. patent application Ser. No. 11/279,068, titled “System, Method and Computer Product for Simplified Instrument Control and File Management”, filed Apr. 7, 2006, which is hereby incorporated by reference herein in its entirety for all purposes.

Scanner 100: Labeled targets hybridized to probe arrays may be detected using various devices, sometimes referred to as scanners, as described above with respect to methods and apparatus for signal detection.

An illustrative device is shown in FIG. 1 as scanner 100. For example, scanners image the targets by detecting fluorescent or other emissions from labels associated with target molecules, or by detecting transmitted, reflected, or scattered radiation. A typical scheme employs optical and other elements to provide excitation light and to selectively collect the emissions.

For example, scanner 100 provides a signal representing the intensities (and possibly other characteristics, such as color that may be associated with a detected wavelength) of the detected emissions or reflected wavelengths of light, as well as the locations on the substrate where the emissions or reflected wavelengths were detected. Typically, the signal includes intensity information corresponding to elemental sub-areas of the scanned substrate. The term “elemental” in this context means that the intensities, and/or other characteristics, of the emissions or reflected wavelengths from this area each are represented by a single value. When displayed as an image for viewing or processing, elemental picture elements, or pixels, often represent this information. Thus, in the present example, a pixel may have a single value representing the intensity of the elemental sub-area of the substrate from which the emissions or reflected wavelengths were scanned. The pixel may also have another value representing another characteristic, such as color, positive or negative image, or other type of image representation. The size of a pixel may vary in different embodiments and could include a 2.5 μm, 1.5 μm, 1.0 μm, or sub-micron pixel size. Two examples where the signal may be incorporated into data are data files in the form *.dat or *.tif as generated respectively by instrument control and image analysis applications 272 (described in greater detail above) that may include the Affymetrix® Microarray Suite software (described in U.S. patent application Ser. No. 10/219,882, which is hereby incorporated by reference herein in its entirety for all purposes) or Affymetrix® GeneChip® Operating Software (described in U.S. patent application Ser. No. 10/764,663, which is hereby incorporated by reference herein in its entirety for all purposes ) based on images scanned from GeneChip® arrays.

Embodiments of scanner 100 may employ various elements and optical architectures for detection. For instance, some embodiments of scanner 100 may employ what is referred to as a “confocal” type architecture that may include the use of photomultiplier tubes to as detection elements. Alternatively, some embodiments of scanner 100 may employ a CCD type (referred to as a Charge Coupled Device) architecture using what is referred to as a CCD or cooled CCD cameras as detection elements. Further examples of scanner systems that may be implemented with embodiments of the present invention include U.S. patent application Ser. No. 10/389,194, 10/846,261, 10/913,102, and 11/260,617; each of which are incorporated by reference above; and U.S. patent application Ser. No. 11/379,641, titled “Methods and Devices for Reading Microarrays”, filed Apr. 21, 2006, which is hereby incorporated by reference herein in it's entirety for all purposes.

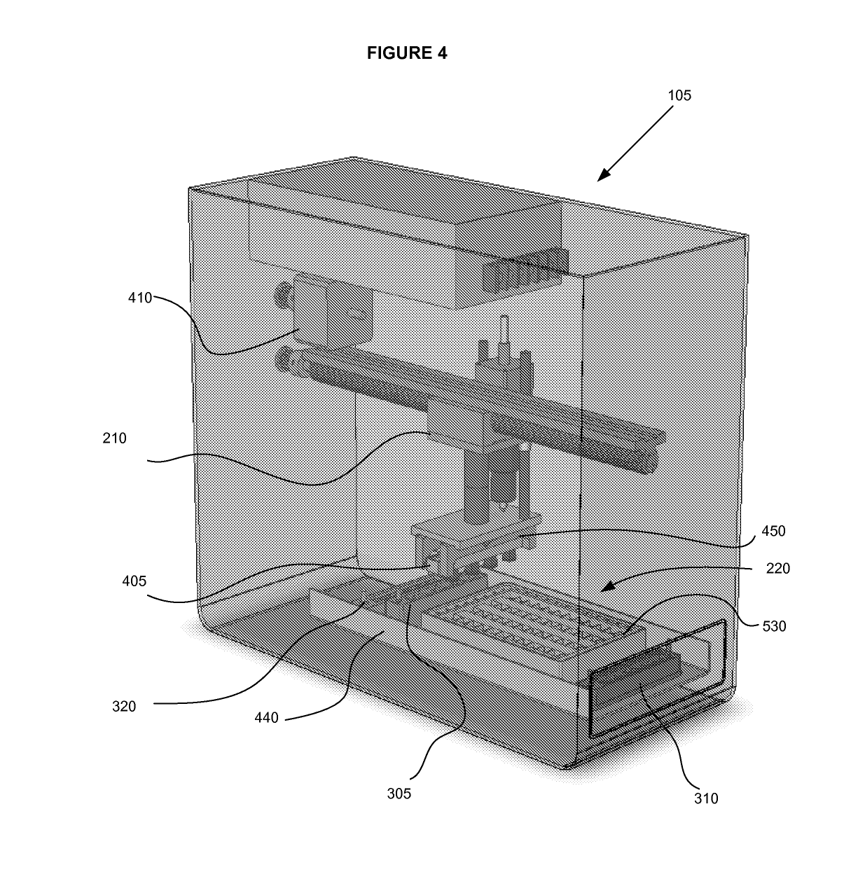

Fluid Processing Instrument 105: Processing embodiments of probe array 140 in preparation for scanning typically involves multiple steps that, when performed manually by user 101, consumes valuable time. FIGS. 3 and 4 provide an illustrative representation of an embodiment of fluid processing instrument 105 comprising stations 220 and robotic manipulator 210 that perform necessary preparation steps without intervention or input from user 101, thus allowing user 101 “walk away” freedom. For example, processing steps for preparing probe array 140 for scanning may include a pre-hybridization step, a hybridization step, a stain step, an antibody step, a plurality of wash steps that may be interspersed between one or more of the previous steps, and one or more other steps as necessary for a particular assay associated with an embodiment of probe array 140. In the present example, the embodiment of fluid processing instrument 105 may be specifically enabled to process multiple embodiments of probe array 140 each for instance associated with peg 505 and affixed to strip 405 as illustrated in FIG. 5A.

FIG. 3 provides a simplified example of stations 220 that may in some embodiments comprise wash station 305, hybridization station 310, stain station 315, scan prep station 320, and antibody station 325. Those of ordinary skill in the related art will appreciate that the number and identification of each of the aforementioned stations should not be construed as limiting and that a greater or fewer numbers of stations may be employed to perform the illustrated or additional method steps. In addition, each embodiment of station 220 may, for instance, be serviced by internal robotic manipulator 210.

As shown in FIGS. 3-5, robotic manipulator 210 may comprise one or more robotic elements enabled to move in the X, and Z axes of a 3-dimensional coordinate system, and comprise one or more elements to operatively couple with one or more trays and/or embodiments of strip 405 for translation between one or more of stations 220. For example, as illustrated in FIG. 4 manipulator 210 may, in some embodiments operatively couple with strip 405 (illustrated in greater detail in FIG. 5A) for the purpose of transporting strip 405 between stations 220 and performing one or more method steps at each station. In the present example, manipulator 210 may include a gripper element 450 to operatively couple with strip 405 (illustrated in FIG. 4 with the embodiments of peg 505/probe array 140 facing downward). Embodiments of a gripper element 450 may comprise elements that reversibly couple with or “grip” the embodiment of strip 405. The gripper element 450 may employ one or more motors to actuate the gripping/releasing actions in cooperation with one or more springs or other elements enabled for these actions. The fluid processing instrument 105 of FIG. 4 can be approximately 12″ H×8″ W×18″ D, for example, or preferably, 11″ H×6 W″×14″ D. It should have a high spill immunity, like the trays 530 themselves, a minimum bench width, and simple to control.

In some embodiments, the gripper element 450 may also include a means of machine readable identification such as, for instance, a barcode reader or other machine readable means previously described. The machine readable means may read one or more machine readable identifiers associated with each of stations 220 for accurate identification and verification that the appropriate station is in its appropriate location and/or includes the appropriate fluid, reagents, etc. for the processing steps.

One alternative arrangement for the barcode reader could include mounting the barcode reader to the gripper element 450 and its associated support and movement hardware just inside the front panel with the reader beam aimed through a window in the front panel. This arrangement allows the barcodes of items on the drawer to be read as the drawer is drawn back into the system. With the drawer fully retracted, the barcode reader can be used to manually scan items. One advantage of this arrangement is that a single barcode reader can be used to manually scan items for experiment setup as well as be used to verify proper loading of the drawer for the protocol being run. The alternative is to have multiple barcode readers, internal and external.

Motor 410 or one or other elements may also enable manipulator 210 to move or translate along an axis relative to drawer 440 that may include an axis of movement that is substantially parallel the primary axis of drawer 440 (presently referred to as the X axis). Drawer 440 can be 12″ long or preferably 9″ long. The load drawer 440 is preferably driven by the same actuator that is used for moving peg strip 405 during fluidic processing. This eliminates the need for an addition actuator for the drawer.

Also, manipulator 210 may be translated towards drawer away from in what may be referred to as the Z axis relative to drawer 440 (up and down). For example the Z axis motion of manipulator 210 may be employed to reversibly translate an embodiment of strip 405 toward one or more of stations 220 for processing. In the present example, manipulator 210 may immerse probe array 140, extended from strip 405 via peg 505, in fluid associated implementations of stations 220 associated with the drawer 440 and/or repeatedly “dip” probe array 140 into the fluid in any of the wells found in drawer 440. After the processing steps associated with the drawer 440 have been completed, manipulator 210 then may retract strip 405 away from the station for transport to another location or station.

In the same or other embodiments, motor 410 may provide the translation force to extend or retract the drawer 440. For example, some embodiments of fluid processing instrument 105 may include a door, aperture, or other means for allowing drawer 440 to extend from the instrument for the purpose of user interaction or intervention that may include loading trays of solution, buffer or other substance, into one or more positions associated with stations 220. Alternatively, drawer 440 may have a dedicated motor element to perform these tasks.

Mechanical automation platforms used in industrial laboratories require accurate relative position control between a gripper 450, pick-and-place or fluid handling head or gripper element 450 and the drawer 440 or deck of the automation system. Typically, the head-to-deck relative positions are calibrated to account for significant manufacturing variations in the system components. Once calibrated, the positioning system utilizes the stored calibrations to return to calibrated positions or to position new deck locations by interpolation or offset from calibrated positions. It is common for the calibration process to be performed using a mechanical alignment between the head and the table or by visual alignment between the head and the table. Once calibrated, the calibration values are fixed until the manual calibration process is repeated.

Typically, it is very important to know the exact position of drawer 440 when in the retracted position within instrument 105 in order to properly register the location of each of stations 220 for processing steps. For example, the exact position of each of stations 220 needs to be identified so that manipulator 210 can accurately align each probe array 140 with the appropriate chamber or well. Misalignment of the probe array/well relationship could result in substantial damage to probe array 140 such as by contact with partitions, walls, or other solid objects not intended to come into contact with probe array 140 possibly resulting in the complete loss of the probe array. Those of ordinary skill in the related art will appreciate that mechanical components such as those associated with drawer 440 are subject to mechanical fluctuation and differences due to environmental conditions and mechanical wear and thus retracted position may vary. An example of compensating for positional variation includes a method of positional identification and associated method. In the present example, drawer 440 may include a plurality of registration marks such as those typically used with what is referred to as a linear encoder. For instance, it may be desirable to associate registration marks in the X and Y axes of drawer 440, where the marks may include reflective elements or other optically identifiable mark. Instrument 105 may include an optical element, such as a linear encoder, that reads the registration marks associated with drawer 440 for position determination. Also in the present example, applications 272 may be employed for determining or calculating the position of drawer 440 that may be advantageous when for instance more complex interpolation calculations must be made to determine the position of drawer 440 within a measure of acceptable accuracy. Additionally, applications 272 may employ the determined position of drawer 440 to generate positional commands to direct manipulator to the appropriate locations.

To improve positioning performance and reduce system and service costs, it is desirable to use a system that automatically, and during use, calibrates the relative position from the head or gripper element 450 to any number of table locations. The relative positioning performance over the life and wear of the system is maintained by the in-use calibration system. Calibration errors are reduced by eliminating the subjective nature of visual or mechanical calibration methods that depend on the skill of the technician. Calibration and setup costs are reduced by elimination of manual calibration methods. System costs are reduced by utilizing less expensive positioning components.

The head (gripper)-to-deck (drawer) relative positions of a robot platform may be accurately calibrated with a position sensing system that is integral to the head and is able to detect the deck. Considering that the position and orientation of a free body can be represented by three discrete points, the head-to-deck calibration may be accomplished by sensors to accurately detect the 3-dimensional location of three fiducials located on the deck. After detection of each of the three fiducial locations, all deck locations are known assuming accurate deck machining. Additional fiducials can be incorporated into the deck at critical deck locations. Also, tools with integral fiducials may be periodically located at important deck locations and detected by the position sensors. Each calibrated deck location can be stored in non-volatile memory for subsequent repositioning. This approach allows the positioning system to take into account manufacturing variations as well as variations over the life of the system. Robot head orientation may also be detected and calibrated by incorporating multiple sensors into the head assembly. Assuming sufficient degrees of freedom of motion for the head, orientation positional errors, such as roll, pitch, yaw, can be corrected for after detection of deck fiducials.

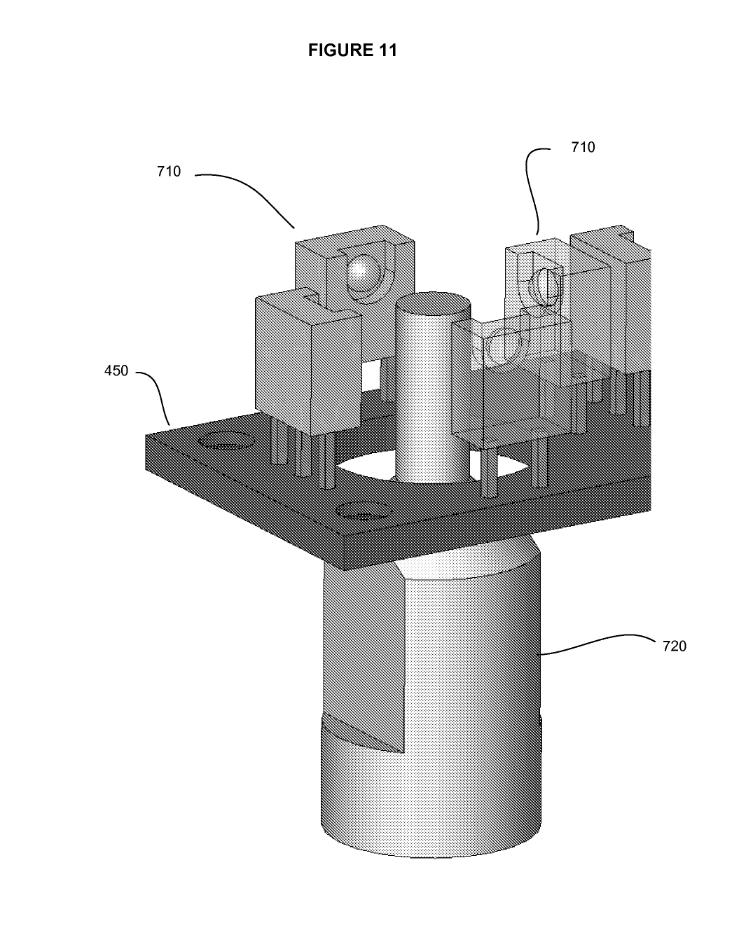

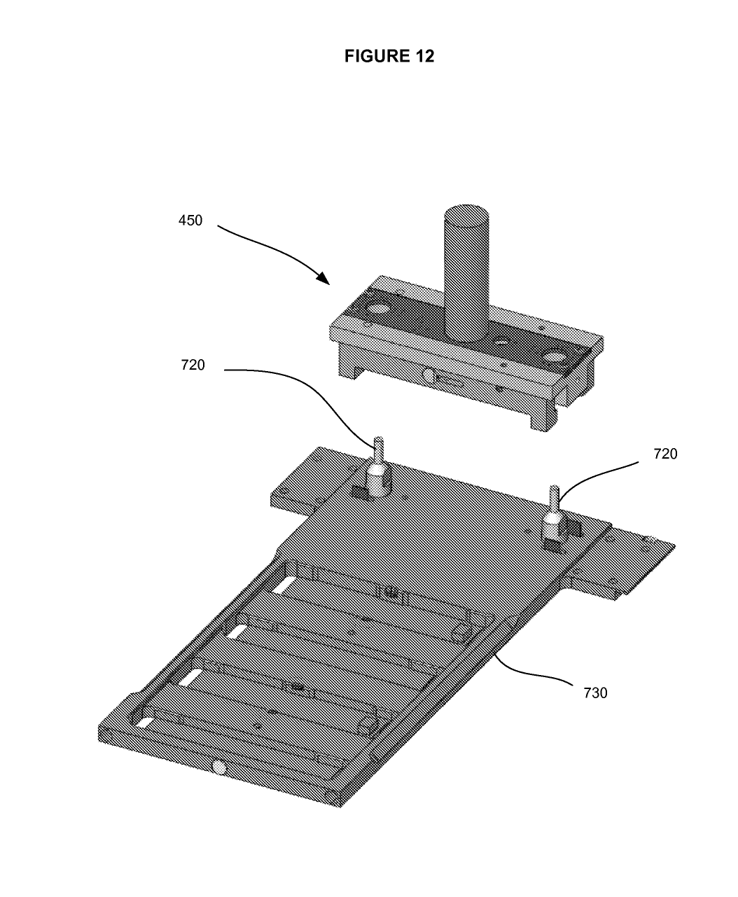

One preferred detector for a positioning system is a photoelectric slot sensor. They are commonly utilized to establish an absolute positional reference for robot systems. These sensors are inexpensive and are highly repeatable and are commercially available from companies such as Balluff, Datasensor, Optex, and Inprox among others. Slot sensors generally detect objects that pass between two arms-one with the emitter, the other with the receiver. The fixed slot width provides reliable opposed-mode sensing of objects as small as 0.30 mm. A pair of photoelectric sensors placed orthogonal to each other may be used to detect the edges of a pin in each of the X, Y and Z directions, where the Z edge is detected by the same sensor as for X or Y. See FIGS. 11 and 12 showing an implementation of two pair of discrete optoelectronic sensors 710 positioned around a fiducial pin 720. FIG. 12 shows the gripper element 450 as it approaches the fiducial pins 720 on a support 730 that can hold well containers similar to the drawer 440. In a preferred embodiment, the gripper element 450 registers its position relative to the support 730 multiple times during the handling operation. Preferably, the position will be registered between each fluid handling step.

Also, some embodiments of instrument 105 may include a means of machine readable identification capable of reading a machine readable identifier associated with each of stations 220 as the drawer 440 retracts thus translating the identifiers past the means for reading. Such an implementation may be included in instrument 105 in addition to or instead of the means associated with the gripper element 450 described above.

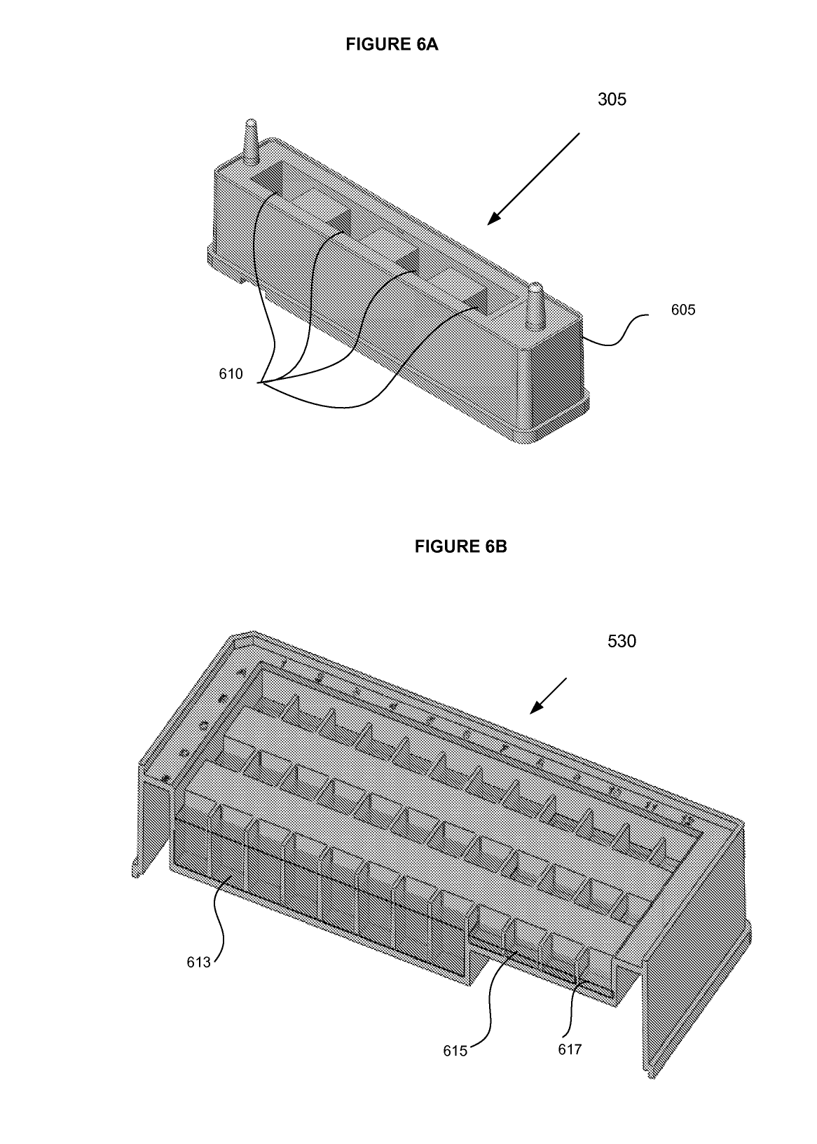

As shown in FIG. 6A, embodiments of wash station 305 may include wash tray 605 comprising a compartment such as chamber 610 associated with each peg 505 and array 140 associated with strip 405 for the purpose of performing “washing” steps with buffers, reagents, or other solutions. In some implementations one or more of chambers 610 may be temperature controlled to provide desirable conditions for one or more processing steps.

For example, some processing protocols for particular embodiments of probe array 140 may call for what may be referred to as a “Wash-B” step or what may be referred to as a high stringency step, where the temperature of the wash-B solution during the processing step could affect the results obtained from probe array 140. A “Wash-A” step can include more standard room temperature wash in a standard buffer. Wash A and wash B are sold commercially by Affymetrix, Inc., Santa Clara, Calif. For more information on Wash A or B, see chapter 4 of the Expression analysis manual at http://www.affymetrix.com/support/downloads/manuals/expression_analysis_technical_manual.pdf. In the present example, the temperature of the wash-B solution during the processing step may preferably be in the range of 36-44 degrees C., and more preferably in the range of 38-40 degrees C. Also in the present example, wash tray 605 may be configured to couple with a heat block where for instance tray 605 may fit over the heat block such that the heat block surrounds each chamber 610. Thus, the heat block provides efficient heat transfer to the fluid contained within each of chambers 610. The heat block may be larger on the ends so that the center wells do not become hotter than the edge wells.

A temperature sensor may be incorporated in the heat block to control the heater for the heat block. There may be a temperature difference between the heat block and the liquid in the wash tray 605. The liquid may be cooler due to heat loss to the room temperature (RT) air surrounding the wash tray. The heat block temperature may be set higher to make up for this difference. The amount it is set higher may be adjusted based on RT. A look up table may be programmed based on the measured RT and setpoint temperatures. A calibration may be performed on individual wash stations 305 to generate this look up table.

The same tray may be used in other process steps. One example of the order of processing steps can be a prehybridization step, hybridization, Wash B, Wash A, stain and an antibody step.

An example process flow can be as shown in Table 1 below. Variations of the times below are within the skill of the art.

| Process Step | Time | Temp |
|---|---|---|
| Peg strip in ship container | — | RT |
| Prehybridization buffer | 20 min. | RT |
| (used in expression exps.) | | |
| Hybridization | 16 hours | 48-60° C. |
| Wash A | 3 min | RT |
| Wash A | 3 min | RT |
| Wash B | 25 min | 42° C. |
| Stain | 10 min | RT |
| Wash A | 3 min | RT |
| Wash A | 3 min | RT |
| Antibody | 10 min | RT |
| Wash A | 3 min | RT |
| Wash A | 3 min | RT |
| Stain | 10 min | RT |
| Wash A | 3 min | RT |
| Wash A | 3 min | RT |
| Scan | | |

Additional wash steps may also be performed that do not require a high degree of temperature control, where for instance the ambient temperature within the instrument environment may provide acceptable results. Multiple use tray 530 may, in some embodiments, include wash wells 613 arranged in rows where each row comprises an implementation of well 613 for each implementation of peg 505 and array 140 associated with strip 405. For example, wells 613 may contain fluids such as one or more wash solutions, buffer solutions, or other solution called for by the processing step. Tray 530 may provide advantages because it is relatively inexpensive and has no mechanical pumping elements or valves, it consumes low volumes of reagent, is less prone to spilling or leakage, and is reusable. As described above, robotic manipulator 210 may translate strip 405 along a Y axis into a row of wash wells 613 where each row is associated with a particular processing step. Typically, each peg 505 embodiment is positioned so probe array 140 faces down over the wells of tray 530. Manipulator 210 may then translate strip 405 along the Z axis to “dip” the embodiments of probe array 140 into the solution present in tray 530. The process of dipping and retracting may be repeated iteratively as defined by the method or protocol process until complete, where manipulator 210 may retract strip 405 away from tray 530 and along the y axis for transport to a different embodiment of station 220. The trays should be held down to prevent pickup during the movement of the strip 405.

As described above, other embodiments of station 220 may include hybridization station, stain station, scan prep station, and antibody station that each may have one or more specialized elements such as trays enabled to carry out or prepare for one or more processing steps. For example, stain station 315 may also be associated with multiple use tray 530, illustrated in FIG. 6B. The trays 530 can include was wells 613, stain wells 615, and antibody wells 617. Each of wells 615 may include one or more stains such as what may be referred to as R-phycoerytherin, CY3, CY5, fluorescein, one or more species of semiconductor nanocrystal (sometimes referred to as “Quantum Dots”), or other type of label known to those in the related art for identifying a target molecule. Manipulator 210 may dip embodiments of probe array 140 in the same manner as described above into wells 613, 615, and 617. Also, certain embodiments of stain are expensive and do not require the greater volumes that are advantageous for the wash processing steps (i.e. greater volume advantageous for dilution of material washed from probe array 140). Therefore, stain wells 615 may include a reduced depth in comparison to wash wells 613 to reduce unnecessary reagent volume and cost. Similarly, antibody wells 617 may also comprise reduced depth for similar reasons. Alignment pins may be added.

In the same or other example, scan prep station 320 may include one or more specialized elements that could for instance include a scan tray that has an optically clear bottom that allows for excitation and emission light to pass. Manipulator 210 may position strip 405 in a corresponding scan tray that may operatively couple with strip 405. For example, strip 405 may comprise one or more alignment features 520 and/or one or more engagement features 510 (FIG. 5A). The specialized scan tray 320 also comprises complementary features such that the features of the scan tray and features 510 and 520 operate to accurately align and secure the tray and strip 405. Drawer 440 may then be translated out to allow user 101 access to the coupled strip 405/scan tray embodiment that may be transported by user 101 to scanner 100 for image acquisition. In the present example, the scan stray may be filled with a solution such as a buffer solution that envelopes and fills the space between probe array 140 and the optically clear bottom or window to reduce optical distortion effects caused by various effects such as the index of refraction where the index of refraction between the window/buffer interface may be less than the index of refraction between a window/air interface and therefore may be more desirable.

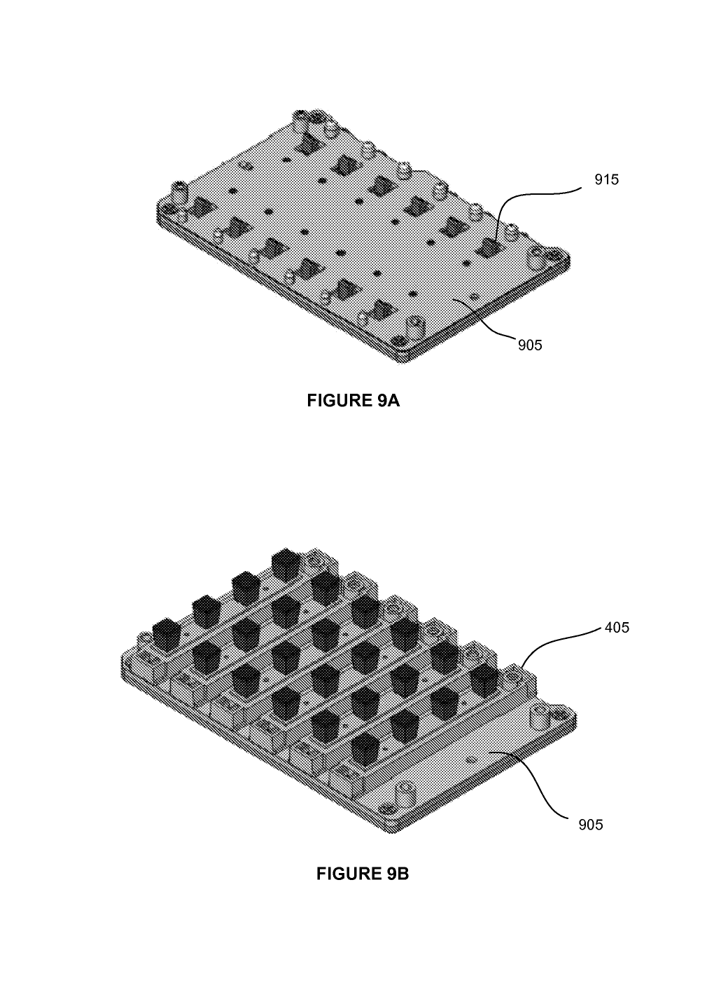

Also in the same or other embodiments, stations 220 may include hybridization (hyb) station 310 that may include a hybridization tray 310 (FIG. 5B) that operatively couples with strip 405 in a similar manner as described above with respect to the scan tray 320. Manipulator 210 may also transport strip 405 coupled with the hyb tray embodiment and similarly drawer 440 may be translated so that the hyb tray is accessible to user 101. User 101 may transport the coupled strip 405/hyb tray embodiment to an external hybridization station that provides one or more environmental control features such as, for instance, temperature and/or humidity control that provides a substantially optimal environment for the hybridization process the efficiently occur. One preferred embodiment includes a hybridization tray in which as many as six strips of pegs slide in from one side and are held in place with fasteners (see FIGS. 9A and 9B). Preferred fasteners include clips, clamps, snaps, latches, slotted joints, flanges, shank apertures, screws and the like.

Additional examples are described in U.S. Provisional Patent Application Ser. No. 60/747,690, titled “Consumable Elements for Use with Fluid Processing and Detection Systems”, filed May 19, 2006, which is hereby incorporated by reference herein in its entirety for all purposes.

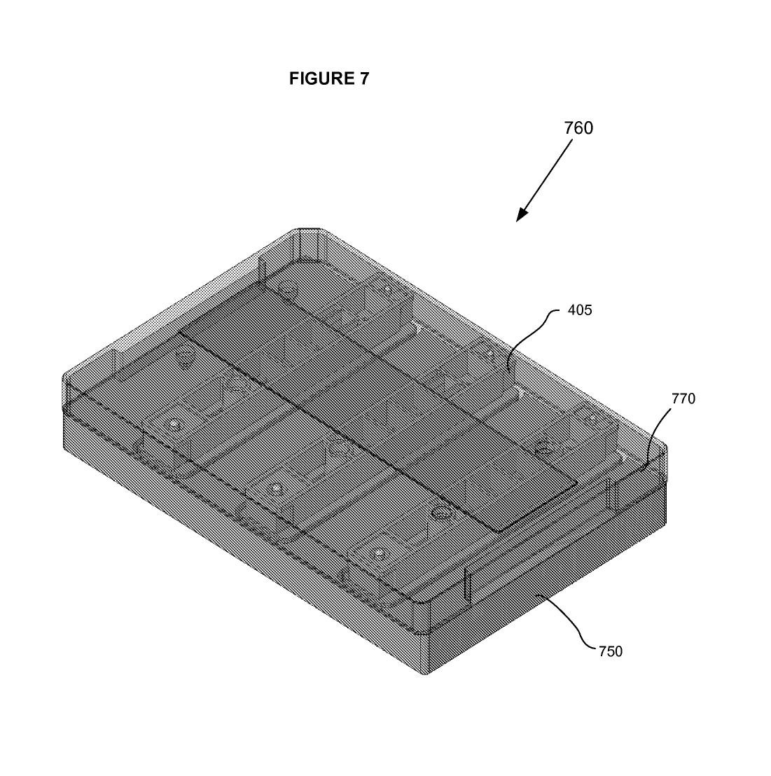

The fluid processing instrument 105 may be coupled to a hybridization station and scanning station for convenient positioning of the individual elements of the system. As shown in FIG. 7, a tray 750 can hold the strips 405 while they are scanned. Multiple strips 405 are held while scanning. Preferably, 2, 3, 4, 5, up to 10 strips may be held in a device such as that shown in FIG. 7. One preferred embodiment 760 uses a cover for the tray while holding the strips 405. Other convenient shapes or embodiments can be used.

Tray 530 can be set for 96 wells or any other convenient arrangement. For example commercial microtiter plates are available in 96, 384, and 1536 wells. 9 mm pitch (distance between wells) is preferred for 96 well plates. The plates can be constructed of many types of durable materials, for example hard plastics like Lexan HP1-112 or polycarbonate, to name a few. They should be structurally sound and machineable within the appropriate engineering specifications. The finished parts should be free of defects like splay, sink marks, scratches and wild lines. No contaminating lubricants or other chemicals should be in the well cavity to affect the hybridization. Smaller trays such as the wash station 305 should be similarly constructed.

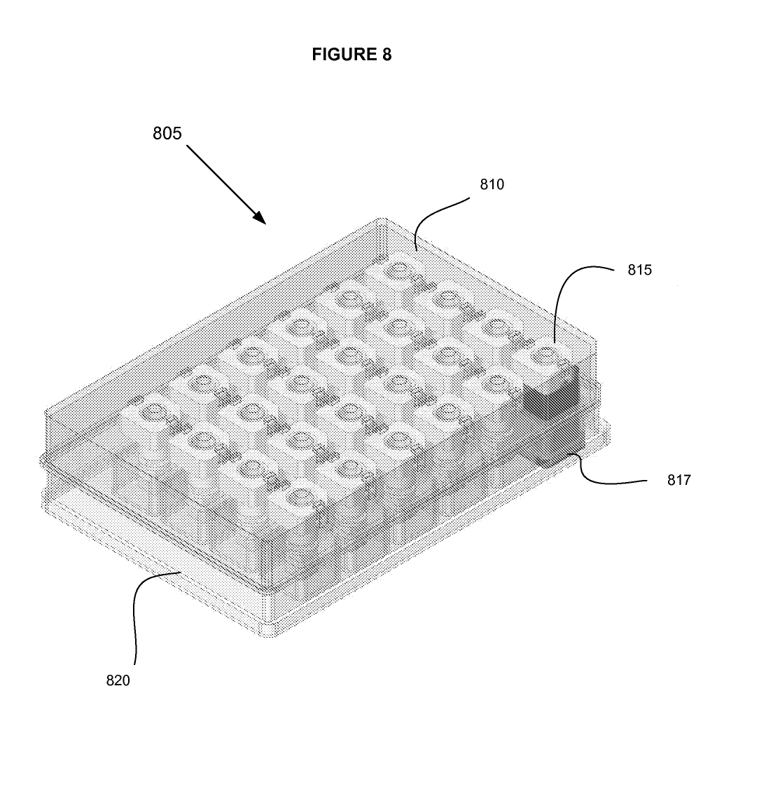

Flexibility is also possible with a device that can accommodate single pegs with associated hybridization trays. FIG. 8 shows a device 805 in which a top plate 810 can hold as many as 24 pegs, although a device may be constructed which can hold more or less pegs. For example, up to 50 or as low as 5 pegs. A user can insert individual pegs 815 with single hybridization trays 817 up through the bottom plate 820. When the top plate 810 is removed from the bottom plate 820, the hybridization trays 817 are left behind. The top plate 810 orients and snaps onto the top of a peg 815.

A device may be employed to hold peg strips 450 together to be processed as a unit.

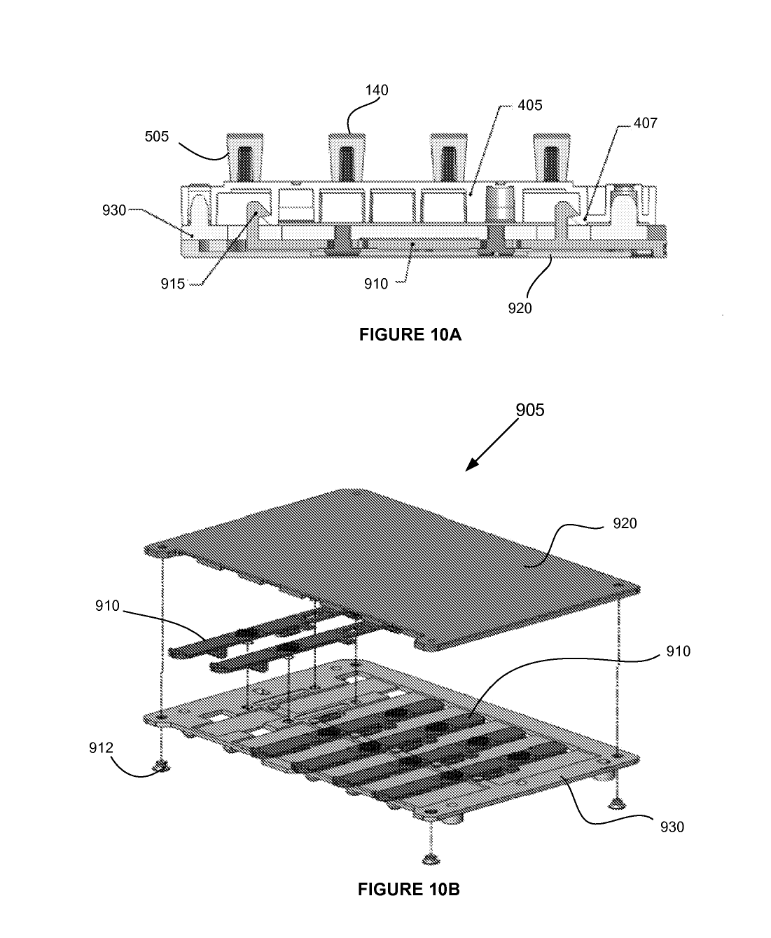

FIGS. 9A, 9B, 10A and 10B show an adapter plate 905 complete with a snap fit bracket 910 for the attachment of individual strips 405. FIG. 9A shows the adapter plate without the strip 405 and FIG. 9B shows that a strip 405 can be attached to the adapter plate 905. FIG. 10A shows a cross sectional view of the adapter plate 905/peg strip 405 combination. FIG. 10B shows an inverted, exploded view of the adapter plate 905.

FIGS. 9A, 9B, 10A and 10B show one preferred embodiment of the adapter plate 905 and the peg strip 405 which are designed such that the peg strip 405 can be assembled to the adapter plate 905 via a snapping mechanism 910. This assembly provides various configurations of 4, 8, 12, 16, 20 and 24 peg arrays on footprint of standard microtiter plate. The peg array assembly can then be scanned in the Affymetrix® 96-frame scanner or high throughput scanner.

As shown in FIGS. 10A and 10B, the adapter plate 905 consists of three parts: the base plate 920, the snap-fit bracket 910 and the lid 930. The base plate 920 and lid 930 can be made of aluminum and the snap-fit bracket 910 can be made of stainless steel, among other durable materials. The snap-fit bracket 910 has two slots and two snaps 915. It is attached to the base plate 920 with two precision shoulder screws 912 and constrained by an extension spring (not shown). The peg strip 405 is assembled to the adapter plate 905 using the downward force. During the assembly process, the snaps 915 located on the bracket 910 are moved back to clear for the snap 407 on the peg strip 405 to pass and the spring is extended.

Once the snaps 917 and 407 are engaged, the spring retracts and applies spring force to keep the snaps fastened. The peg strip 405 is then held in place securely to the adapter plate 905. The peg strip 405 can be disassembled from the adapter plate 905 by pushing against the end handle of the snap-fit bracket 910 to disengage the snap-fit.

The adapter plate 905 and the peg strip 405 each have their own snap features. The peg strip 405 is assembled to the adapter plate 905 via the snap lock mechanism 910 established by the snap features. The snap fit bracket may contain a snap 915 on the bracket to connect with a snap 407 on the strip 405. Other methods of attachment can be used such as clamps, toggles, screws and other fasteners known to those of skill in the art. In the embodiment shown in FIG. 9B, six strips 405 are attached to the adapter plate 905. However, fewer strips 405 may be affixed and plates 910 may be designed to accommodate more strips 405.

A prototype peg strip 405 was made of SL7520 rigid resin using stereo lithography process. A prototype adapter plate 905 was made of aluminum using CNC machining process. The prototype demonstrated that the adapter plate 905 provided the secure fastening of peg strips 405 for multiple peg array configuration.

Having described various embodiments and implementations, it should be apparent to those skilled in the relevant art that the foregoing is illustrative only and not limiting, having been presented by way of example only. Many other schemes for distributing functions among the various functional elements of the illustrated embodiment are possible. The functions of any element may be carried out in various ways in alternative embodiments.

Also, the functions of several elements may, in alternative embodiments, be carried out by fewer, or a single, element. Similarly, in some embodiments, any functional element may perform fewer, or different, operations than those described with respect to the illustrated embodiment. Also, functional elements shown as distinct for purposes of illustration may be incorporated within other functional elements in a particular implementation. Also, the sequencing of functions or portions of functions generally may be altered. Certain functional elements, files, data structures, and so on may be described in the illustrated embodiments as located in system memory of a particular computer or instrument. In other embodiments, however, they may be located on, or distributed across, computer systems, instruments, or other platforms that are co-located and/or remote from each other. For example, any one or more of data files or data structures described as co-located on and “local” to a server or other computer may be located in a computer system or systems remote from the server. In addition, it will be understood by those skilled in the relevant art that control and data flows between and among functional elements and various data structures may vary in many ways from the control and data flows described above or in documents incorporated by reference herein. More particularly, intermediary functional elements may direct control or data flows, and the functions of various elements may be combined, divided, or otherwise rearranged to allow parallel processing or for other reasons. Also, intermediate data structures or files may be used and various described data structures or files may be combined or otherwise arranged. Numerous other embodiments, and modifications thereof, are contemplated as falling within the scope of the present invention as defined by appended claims and equivalents thereto.

What is claimed is:

1. An automated and flexible system for conducting hybridization experiments comprising:
a plurality of pegs holding nucleic acid arrays removably mounted on a strip;
an automated robot for moving a strip to selected location;
at least one tray containing wells in rows, the wells having reagents to interact with an array on the peg, each row being associated with a different process step; and
a system to align the peg with the wells on the tray.

2. The system in accordance with claim 1 where the strip has four pegs.

3. The system in accordance with claim 1 where the strip is removably mounted to a plate.

4. The system in accordance with claim 1 where the strip is encased.

5. The system in accordance with claim 1 further comprising a bar code reader to identify strips or trays.

6. The system in accordance with claim 1 further comprising operatively connections to a hybridization station and scanning station.

7. A system in accordance with claim 1 wherein the system to align the pegs with the wells on the tray, comprises:
a set of fiducial features operatively connected to the tray; and
a set of sensors operatively connected to the automated robot to determine a position for the tray relative to the robot.

8. A system in accordance with claim 1 for controlling the temperature of a wash solution comprising:
a heater block whose shape is tailored to give the same temperature in all the wells;
a temperature probe to measure the heater block temperature;
a temperature probe to measure the room temperature; and
a calibrated look up table to adjust for differences between the heater block temperature and the temperature of liquid in the wells based on room temperature.

9. A system in accordance with claim 1 wherein there is more than one strip that is held together with a system which comprises; a base plate; a snapfit bracket; and a lid.

10. An automated and flexible system for conducting hybridization experiments comprising:
a plurality of pegs holding nucleic acid arrays removably mounted on a plurality of strips, the strips are held together with a system which comprises; a base plate; a snapfit bracket; and a lid;
an automated robot for moving a strip to selected location;
at least one tray containing wells in rows, the wells having reagents to interact with an array on the peg, each row being associated with a different process step, comprising a wash step having a heater block whose shape is tailored to give the same temperature in all the wells; a temperature probe to measure the heater block temperature; a temperature probe to measure the room temperature; and a calibrated look up table to adjust for differences between the heater block temperature and the temperature of liquid in the wells based on room temperature; and
a system to align the pegs with the wells on the tray, comprising a set of fiducial features operatively connected to the tray; and a set of sensors operatively connected to the automated robot to determine a position for the tray relative to the robot.
